SEQUENCE LISTING (22 sequences)

SEQ ID NO: 1 - protein, 594 aa, Arabidopsis thaliana
MRDDKTKKSKLSWSKKMVRKWFNIKSKTEKFQADVSLPQGVEVEHRNSFSEREPCTIKKSKTEKLNKNWEQQARQRKMNYENPRIIDVQNHSIFVATWNVAGRSPPEDLNLDEWLHSSAPADIYVLGFQEIVPLNAGNVLGAEDNGPAKKWHSLIRKTLNNLPGASSACHTPSPIPVPIAEIDADFSGSSRQKNETFFNRRSFQTPSVWSMEENDPSISQPRLDRRFSVCDRVFFSHRPSDFDPSFRCGHRPSDYSRRPSDYSRPSDYYSRPSNYSRPSDVSRWGSSDDDNGPGDSPSTFLNSPGSFLGSAANENGYRTPWNSSQYCLVASKQMVGIFLTIWVKSELREHVKNMKVSCVGRGLMGYLGNKGSISISMLLHQTSFCFVCTHLTSGQKEGDELRRNSDVMEILKKTRFPRVQSSADEKSPENILQHDRVIWLGDLNYRIALSYRSAKALVEMQNWRALLENDQLRIEQKRGHVFKGWNEGKIYFPPTYKYSNNSDRYAGGDLHPKEKRRTPAWCDRILWHGEGLHQLSYVRGESRFSDHRPVYGIFSAEVESNHKRSKRTNSHSTARVEAEELLPYARGYTELTFF

SEQ ID NO: 2 - DNA, 2686 bp, Arabidopsis thaliana
atcctctgcttttactttgccgtttcactgagaacgaggctcgctcactcactctctctctttctctttctctttctctttctctttcatagaagaagaagaagattcaggacctaagtagaaaagcttaaaccaccactctctcttcaaatcacacttttgtttgtttcctctctctccaaaatctttctcattgtcttctttgcctcctccttcaagaactcttgtctcttccaacaacttttttccttcttattctgaaaaccaatcttcacttctcatttgtctcaaaacactatacccatttcctaattcctgtccctgagagcattagtagagagacacaaaacactttaatcctaatccaacttgtctttactttagcttctgtctgattcaatcaaaggacaagaaagaagccaaatcttgtcaattctttttaaacagtgattcaactaaaaaccccaaattagttgatttgatctctattgtcttcttttttttggatggattgagtctcaagaacaaacagtgaatcaaataagagcaagaatgagagacgataaaaccaagaaaagcaagttgtcatggtccaagaagatggtgaggaaatggttcaacatcaaaagcaaaactgaaaagtttcaagcagatgtttctcttcctcaaggagttgaagttgagcacagaaacagcttctcagagagagagccctgcacaatcaagaagagcaaaactgaaaaactcaataagaactgggagcagcaagctagacaaaggaaaatgaactatgaaaatcccagaatcattgatgtccaaaaccacagcatcttcgtagctacatggaacgttgctggacgctctcctcctgaagatttaaacctcgacgaatggcttcattcttcagctcctgcagatatatacgttctcgggttccaagagattgttcctcttaatgctggtaatgttcttggagctgaagacaatggtcctgctaagaaatggcattcactcatcaggaaaacgctcaataaccttcccggagcaagcagtgcatgccatactccatcgcctattcctgtccccattgcagagattgatgctgacttctcaggatcttcaaggcaaaagaacgagactttcttcaacagaagatctttccagacgccgagcgtatggagtatggaggagaatgacccttcgatctcacaaccacggcttgaccgtcgtttcagcgtgtgtgatcgcgtattcttcagtcatagaccaagtgattttgacccgagcttcaggtgtggtcacaggcctagtgattactcaagaaggcctagtgattactctagaccgagtgattattactctaggccgagtaactattctcggcctagtgatgtttccaggtggggctcctcggatgatgataacggcccaggggattctccaagcacatttttgaattcaccggggtcatttcttggttctgcagcaaacgaaaacggttacagaaccccgtggaactcatcgcagtactgtttggtcgcaagtaaacagatggttggtatctttttaaccatttgggtgaaaagcgagttacgagaacacgtcaagaacatgaaagtatcttgtgttggtagaggattaatgggttatctcggaaataagggatcaatctcaattagtatgttgcttcatcaaacaagcttctgctttgtgtgcacccatttgacctcgggacaaaaagaaggtgatgagctaaggagaaactccgatgtcatggagatactcaagaaaacaaggtttcctcgagtccagagttctgcggacgagaagtcccctgagaatatccttcagcatgatcgggtaatatggctcggggatctgaactaccgaatagcactctcttaccgatccgcaaaagcacttgtcgagatgcaaaactggagggcattactagagaacgatcagttacggatagagcagaaacgaggacatgtgtttaaaggatggaacgaaggaaagatatacttcccaccaacttacaaatactcaaataactcggatagatatgcaggaggtgatttgcatccaaaggagaaacgtcgtactcctgcttggtgtgatcgaatactatggcatggagaaggactacatcagttatcttatgtaagaggggagtcacggttttctgatcatagaccggtttatggcatattcagcgcagaggttgagtcaaatcacaagagatcaaagcgaaccaatagtcactccactgcacgggtcgaagctgaagaactcttaccttacgcccgcggatacaccgagctaacatttttctaaaagaaaattgaaaagccttcttctttacgccttgaggagtggatgatggagacatttcattgctccttttaacacaaaggaaacaaataacaggtgtataaaggattgtaattttgtcttggttaatagaatttcgcatatcaaaccggttctttcatttatcatgtcaggagtaggagttaaaagtaaattttacagtgtaggattattaaactgacagatggaaggttttttttagttcttcatcagtctttttagggatagtttttgataatattattattgtaaattgaagtgtgttcccgagacaatgatagtgtaatgcttgttgtataagtaagatttagcc

SEQ ID NO: 3 - protein, 417 aa, Arabidopsis thaliana
MWPRLVANKILRKSLGSNNFVADFPPNTDQKLIEASGLADERSKSILHNQHKTTLLNYKVFVSTWNVGGIVPDDGLDMEDLLETHKTPCDIYVLGFQEVVPLRASNVLGSDNNKVSTKWNSLIRDALNKRARPHRDEDLSESKGINGISQDFRCIISKQMVGILITVWVRGDLWPYIRYPSVSCVGCGIMGCLGNKGSVSVRFQLHETTFCFVCSHLASGGRDRDERQRNSDVNEILARSSFPRGSSLDLPKKILDHDRVIFLGDLNYRISLPEEKTRLLVESKKWNILLENDQLRMEIMNGQIFRGWQEGIVKFAPTYKYVPNSDLYYGCITYKKDEKKRAPAWCDRIIWYGNGLKQHEYTRGETKISDHRPVKAIFTTEITVTRRGKKIRNFFFSDRFEERIGDIDSKDYSWIST

SEQ ID NO: 4 - DNA, 1553 bp, Arabidopsis thaliana
ttctttctatctcttgttctttaatttgtctctctctttttctctagcaatgcttggaagctatagagaagtgagtattagtgtctctctctttactttcactatctctgtttagatgtgtgtgtcggatatataaccggactaatatacgtataacttatagttattctatcttgaccattgatatatgcgaatcactctatggtattaataaggttatgtggccaagacttgtggccaacaagatcctaaggaaaagcctcgggagcaacaacttcgttgccgatttcccacctaatactgatcagaagcttatagaagcttccggattagctgatgaacgatctaaatcaattctccacaaccaacacaaaaccactcttctcaactacaaggtttttgtaagcacatggaacgtgggtggtatcgtaccggacgatggacttgacatggaggatctactggaaacacataaaacaccgtgcgacatatacgtgcttgggtttcaagaggttgtgcctctacgagcttcgaatgttttgggatcagataacaacaaagtctctacaaaatggaactctttgataagagatgctttgaacaagagagcaagacctcatcgagacgaagacttgtcggaatcaaagggcattaatggtatttcacaagattttagatgtatcataagtaaacagatggtcggaatcttgatcaccgtttgggtccgaggtgatctctggccgtacatccgttatcctagcgtttcatgcgttggttgcggcatcatgggttgcttaggaaacaagggatcagtatcggttagatttcaattgcacgaaacgacattttgtttcgtttgtagccatctagcttctggtggccgcgaccgagatgaaagacagagaaactccgacgtcaacgagatcttagctagatctagttttcctcgaggctcatctctagatttacccaaaaagatccttgatcacgatcgggtgatttttttgggagacttgaattatagaatttcattaccggaggagaagacaagattattggtggaaagcaaaaaatggaacatcttactagaaaatgatcagctgaggatggagatcatgaacggccagattttcagagggtggcaagaaggaattgtcaaatttgctccaacgtataaatacgttccaaattcggatttatattacggatgcattacatacaaaaaagacgagaagaaacgagctcctgcatggtgtgatcgaataatatggtacggaaatggactgaagcaacatgaatatacaagaggagaaacgaagatatctgatcataggcctgtcaaagcaattttcaccacagaaattactgtaacacggcgtggtaaaaagattcgtaatttctttttctcggacagatttgaagaaagaattggtgacatagattctaaagactactcgtggatatctacatagttttattaatcggggtgatatcttgtcacttgttaatatacaattcacatagattttatataaaaaaaacttatacatatg

SEQ ID NO: 5 - DNA, 24 bp, Artificial Sequence
  Synthetic Polynucleotide
gccatggtggtgattcttgagaac

SEQ ID NO: 6 - DNA, 23 bp, Artificial Sequence
  Synthetic Polynucleotide
gaaaaatgttagctcggtgtatc

SEQ ID NO: 7 - DNA, 28 bp, Artificial Sequence
  Synthetic Polynucleotide
caccatgagagacgataaaaccaagaaa

SEQ ID NO: 8 - DNA, 24 bp, Artificial Sequence
  Synthetic Polynucleotide
ttagaaaaatgttagctcggtgta

SEQ ID NO: 9 - DNA, 24 bp, Artificial Sequence
  Synthetic Polynucleotide
ctgcttggtgcaagtgctgtgatt

SEQ ID NO: 10 - DNA, 24 bp, Artificial Sequence
  Synthetic Polynucleotide
agaagtcttgttccagccctcgtt

SEQ ID NO: 11 - DNA, 24 bp, Artificial Sequence
  Synthetic Polynucleotide
cggtgatcgaacacttcaactcca

SEQ ID NO: 12 - DNA, 24 bp, Artificial Sequence
  Synthetic Polynucleotide
ttcgattctgatcgttacgccgga

SEQ ID NO: 13 - DNA, 24 bp, Artificial Sequence
  Synthetic Polynucleotide
atcgctgtaaccggacgatgatct

SEQ ID NO: 14 - DNA, 24 bp, Artificial Sequence
  Synthetic Polynucleotide
ttccgatgagtacaccgcaaacga

SEQ ID NO: 15 - DNA, 21 bp, Artificial Sequence
  Synthetic Polynucleotide
tcagaaaaccgtgactcccct

SEQ ID NO: 16 - DNA, 24 bp, Artificial Sequence
  Synthetic Polynucleotide
gatggaacgaaggaaagatatact

SEQ ID NO: 17 - DNA, 24 bp, Artificial Sequence
  Synthetic Polynucleotide
acccacttcaagcaaagatccgct

SEQ ID NO: 18 - DNA, 24 bp, Artificial Sequence
  Synthetic Polynucleotide
gccataagatcagggttccagctt

SEQ ID NO: 19 - protein, 441 aa, Arabidopsis thaliana
MSTTIRFLRIVTSRAMSSSWSCNKCTFLNSASQKLNCMICLAPVSLPSLSPPPPSLSISANDEAKWACKACTFLNTYKNSICDVCGTRSPTSSLLGFQDLTDSGLESNDADSSVGSVFFPLRRCIKRKAMDDDVVEVDGASVVCSESQGVMKKNKEIETKGVASDSGTPLTCLKILSYNVWFREDLELNLRMRAIGHLIQLHSPHLICFQEVTPEIYDIFRKSNWWKAYSCSVSVDVAVSRGYYCMLLSKLGVKSFSSKSFGNSIMGRELSIAEVEVPGRKPLVFATSHLESPCPGPPKWDQMFSRERVEQAKEAIEILRPNANVIFGGDMNWCDKLDGKFPLPDKWVDVWEVLKPGDLGFTYDTKANPMLSGNRALQKRLDRILCRLDDYKLGGIEMVGKEAIPGLSYVKEKKVRGDIKKLELPVLPSDHFGLLVTLSPK

SEQ ID NO: 20 - DNA, 1648 bp, Arabidopsis thaliana
aaaatcgataaaaactaattagggtaaatgtcaacaacaatccgatttctgagaattgtcacaagtcgagcaatgtcgtcgtcttggtcctgcaacaaatgtactttcctcaattctgcgtctcagaaactcaattgcatgatctgcttggctcctgtatctctcccttctttgtctcctcctccaccttctctctcaatttccgcgaacgatgaagctaaatgggcttgcaaggcctgtacttttttgaacacgtacaagaactcaatctgtgacgtctgtggaacacgatcgccaacgtcttctctcttgggatttcaggatttgactgattcgggtctcgagagtaacgacgctgattcctccgtcgggtctgttttctttcctctgcggcggtgtatcaagcgtaaagctatggatgatgacgtcgtcgaggtcgatggtgcttctgttgtttgctctgaatcgcaaggtgtcatgaagaagaacaaagagattgaaacgaaaggggtagcttctgattcaggaacacctttaacttgcttgaagatcttgagctacaatgtttggtttcgtgaagaccttgagttgaatctaaggatgagagcgattggtcatcttattcagcttcattctcctcatctcatatgttttcaggaagtcactcctgagatatatgatattttccgcaagtcgaattggtggaaagcgtattcttgttctgtctcagttgatgtggcagtttcgagaggctactactgcatgctgttgagcaagttgggagtgaaatctttcagcagcaaaagctttgggaactcgataatgggaagagaactatctatcgcagaagttgaagttccagggagaaagccacttgtttttgcaactagccatcttgagagtccctgtccaggtcctccaaaatgggaccaaatgttcagtcgagaacgcgttgaacaagccaaggaggcaattgagattctcagacctaatgccaatgtgatattcgggggagacatgaactggtgcgacaagctagatgggaagtttcctttgccagacaaatgggtggatgtttgggaggttttgaaaccaggtgatttgggttttacatatgacacaaaagcaaacccaatgttgtccggtaaccgggcattgcagaaacggttagaccggattttgtgtcgtctggatgattacaagcttggtggaatcgagatggttgggaaagaggccattcctggtttatcatatgtaaaagagaagaaagtgaggggtgacattaagaagttagagcttccggtgttgcctagtgatcatttcggtctgcttgtaaccctttcgccaaaatgagatcaaactattcagagctcgaagttagtcttccttagtgaagcttttgtgactaggttaaaaaactgtatgaaacaccaaatttggtgcgtagggtttctctgtttcgaactgatgataaacagttcttggtttaagttttttttgttttttggtttgggaacggccttatttcctgattcgaactgaaagatcagtcttattgttcaacaatgcccccaccttttgtaatgaagattctgaaactaatcacatattaatatgatcgttaaaaaccagagagattttggtatca

SEQ ID NO: 21 - DNA, 25 bp, Artificial Sequence
  Synthetic Polynucleotide
caccatgtggccaagacttgtggcc

SEQ ID NO: 22 - DNA, 24 bp, Artificial Sequence
  Synthetic Polynucleotide
ctatgtagatatccacgagtagtc

(19) United States
(12) Patent Application Publication
(10) Pub. No.: US 2014/0101789 A1
(43) Pub. Date: Apr. 10, 2014

(54) Plants Tolerant To Abiotic Stress

(71) Applicants: Alex Levine, Mevasseret Zion (IL); Yuval Kaye, Moshave Nitzanei Sinai (IL); Yael Golani, Alfe Menashe (IL)

(72) Inventors: Alex Levine, Mevasseret Zion (IL); Yuval Kaye, Moshave Nitzanei Sinai (IL); Yael Golani, Alfe Menashe (IL)

(21) Appl. No.: 14/106,188

(22) Filed: Dec. 13, 2013

Related U.S. Application Data
(63) Continuation of application No. PCT/IL2012/050210, filed on Jun. 14, 2012.
(60) Provisional application No. 61/497,217, filed on Jun. 15, 2011.

(51) Int. Cl. C12N 15/82

(52) U.S. Cl. CPC C12N 15/8273
USPC 800/278; 800/298; 800/323; 800/320.3; 800/320; 800/320.2; 800/320.1; 800/312; 800/306; 800/322; 435/419

(57) ABSTRACT

The present invention provides genetically modified plants having increased tolerance to environmental abiotic stress, particularly to salt stress and water stress (drought). The tolerant genetically modified plants of the invention include transgenic plants overexpressing at least one inositol polyphosphate 5-phosphatase selected from 5TPase7 5TPase9 and plants having altered expression of the Endonuclease/Exonuclease/Phosphatase (EEP) protein ZEEP1.

### FIELD OF THE INVENTION

The present invention relates to genetically modified plants that are tolerant to abiotic stress, particularly to genetically modified plants having altered expression of at least one of the Endonuclease/Exonuclease/Phosphatase (EEP) protein family that show increased tolerance to abiotic stress, particularly to water and salt stress conditions.

### BACKGROUND OF THE INVENTION

Plants of different species have the ability to adapt to the surrounding environmental conditions, including unfavorable conditions. The adaptation process to the various abiotic stresses, such as cold, heat, water stress (drought) and salinity involves significant changes in gene expression. While it has been established that gene induction is caused by binding of transcription factors that interact with RNA polymerase, recent studies showed that the expression of many stress responsive genes during normal growth conditions is actually suppressed by nuclear factors that act as transcriptional repressors (Kazan K. 2006. Trends Plant Sci 11, 109-112).

The Endonuclease/Exonuclease/phosphatase (EEP) family of proteins comprises a conserved structure domain present in a large number of proteins, including magnesium-dependent endonucleases and phosphatidylinositide phosphatases. These proteins are involved in diverse intracellular signaling processes in different organisms (Dlakic M. 2000. Trends in Biochemical Sciences 25, 272-273; Wang H et al., 2010. EMBO J 29(15), 2566-2576).

Phosphatidylinositides comprise a small fraction of the membrane lipids in plants and other eukaryotes. Phosphatidylinositol (PtdIns) is an anionic glycerophospholipid that is located in the plasma and internal cell membranes. PtdIns along with its phosphorylated derivatives (PtdInsPs) are involved in many important cellular processes, including cytoskeleton organization, regulation of endocytosis and membrane trafficking. The PtdInsPs constitute a family of eight molecules that undergo phosphorylation and dephosphorylation in the hydroxyl group of the inositol ring by specific kinases and phosphatases. Recently, the phosphatidylinositides have emerged as important regulators of signaling processes, especially in mediating abiotic stress responses (Ercetin M E and Gillaspy G E. 2004. Plant Physiol 135, 938-946; Leshem Y et al., 2007. Plant J 51, 185-197); Thole J M and Nielsen E 2008. Curr Opin Plant Biol 11, 620-631; Xue H W et al., 2009. Biochemical Journal 421, 145-156). The PtdIns-mediated signaling is controlled by the phosphorylation or dephosphorylation. Most important among the regulatory enzymes are PtdIns 3-kinase and members of the PtdIns 5-phosphatases (5PTases), belonging to the EEP protein family.

In humans, there are eight characterized 5PTases, which have been divided into four groups, based on sequence homology and in vitro substrate specificity. Yeast contain four 5PTases that are homologous to the human synaptojanin and other group I or II enzymes. The plant kingdom possesses increased amounts of 5PTase genes: the Arabidopsis genome contains 15 5PTase genes, and rice (Oryza sativa) has more than 20 genes.

Biochemically, the 5PTases act as phosphoric monoester hydrolases. The 5PTases are characterized by a conserved catalytic domain of approximately 350 amino acid residues. Two conserved catalytic motifs were identified as essential for 5PTase activity and have been grouped into four types according to their substrate specificity. There are four known substrates for 5PTases: Ins(1,4,5)P₃, Ins(1,3,4,5)P₄, the lipids PtdIns(4,5)P₂, and PtdIns(3,4,5)P₃. Type I enzymes hydrolyze the water-soluble inositol phosphate substrates, namely Ins(1,4,5)P₃ and Ins(1,3,4,5)P₄. The type II 5PTases hydrolyze all four 5PTase substrates, although with varying catalytic efficiency. The type III 5PTases hydrolyze phosphate from substrates phosphorylated in the D3′ position, such as PtdIns(3,4,5)P₃ and Ins(1,3,4,5)P₄. The type IV 5PTases dephosphorylate only phosphoinositols that are part of the membrane lipids, such as PtdIns-3,4,5-P₃, or PtdIns-4,5P₂.

Functional analysis of the four 5PTases from Saccharomyces cerevisiae showed that although they are not essential for viability under normal conditions, they play a role in osmotic stress tolerance. Mutations in the 5PTases in humans have been associated with several diseases, including cancer. A crucial role of 5PTase activity was also shown in the immune response in neutrophils, in the intracellular activation of the NADPH oxidase complex in the phagosomes.

The Arabidopsis 5PTase family is composed of variable length proteins, having from 331 (in At5PTase11) to 1144 (in At5PTase14) amino acids. All of the At5PTases contain the conserved catalytic domain (Zhong R and Ye Z H. 2003. Plant Physiol. 132, 544-555). Some of the At5PTases also have additional domains, such as WD40 repeats or MSP (Motile Sperm protein) domains that function in protein complexes that regulate membrane trafficking by interacting with other proteins. Proteins that are regulated by PtdIns(4,5)P2 include the actin-modifying enzymes profilin, cofilin and gelsolin and proteins that control endosomal vesicle fusion with plasma membrane.

A common physiological response to abiotic stress in plants is the increased generation of reactive oxygen species (ROS), resulting in a secondary oxidative stress, which negatively affects plant growth (Smirnoff F. 1998. Current Opinion in Biotechnology 9, 214-219). The major source of ROS generation in plants (as well as in animals) is activation of the NADPH oxidase complex that reduces molecular oxygen, while generating superoxide radicals. Activation of the NADPH oxidase in A. thaliana plants during salt stress was recently shown to be mediated by PtdIns signaling, similar to the situation in neutrophils (Simonsen A and Stenmark H. 2001. Nature Cell Biol. 3, E179-E182.; Leshem et al., 2007, ibid).

A paper by inventors of the present invention and co-workers published after the priority date of the present application shows that At5PTase7 functions in the plant response to salt stress by regulating ROS production and gene expression, suggesting that At5PTase7 coordinates the above responses during salt stress, and showed that overexpression of the At5ptase encoding gene results in resistance to salt stress (Kaye Y et al., 2011 Plant Physiol. 157, 229-241).

Crop production is affected by numerous abiotic environmental factors with soil salinity and drought having the most detrimental effects. Approximately 70% of the genetic yield potential in major crops is lost due to abiotic stresses, and most major agricultural crops are susceptible to drought stress. In water-limited environments, crop yield is a function of water use, water use efficiency (WUE; defined as aerial biomass yield/water use) and the harvest index (HI; the ratio of yield biomass to the total cumulative biomass at harvest). Water deficit can also have adverse effects in the form of increased susceptibility to disease and pests, reduced plant growth and reproductive failure.

There is a continuing need for, and it would be highly advantageous to have means and methods for improving the resistance of crop and ornamental plants to abiotic stress, particularly to water and salt stress conditions.

### SUMMARY OF THE INVENTION

The present invention provides genetically modified plants having altered expression of at least one Endonuclease/Exonuclease/Phosphatase (EEP) protein that show enhanced tolerance to abiotic stress, particularly to soil salinity and drought (water stress) compared to corresponding unmodified plants. According to certain embodiments the EEP protein is selected from the group consisting of Arabidopsis thaliana phosphatidylinositol 5-phosphatase 7 (At5TPase7), Arabidopsis thaliana phosphatidylinositol 5-phosphatase 9 (At5TPase9), Arabidopsis thaliana ZEEP1, homologs and functional fragments thereof.

The present invention is based in part on the unexpected discovery that the expression of several EEP encoding genes was induced as a result of different abiotic stresses, while the expression of others was suppressed by the same stress. Plant harboring mutants of the stress responsive genes At5TPase7 and At5TPase9 were more susceptible to salt and water stress compared to wild type plants, while transgenic plants overexpressing at least one of these genes showed increased tolerance to the stress conditions. On the other hand, plant harboring mutants of the stress responsive EEP encoding gene designated herein ZEEP1 showed increased tolerance to abiotic stress compared to corresponding wild type plants.

Thus, according to one aspect, the present invention provides a genetically modified plant comprising at least one cell having altered expression of at least one of Endonuclease/Exonuclease/Phosphatase (EEP) protein corresponding to Arabidopsis thaliana 5TPase7 (At5TPase7), Arabidopsis thaliana 5TPase9 (At5TPase9) and Arabidopsis thaliana ZEEP1, wherein the plant has increased tolerance to environmental abiotic stress compared to a corresponding unmodified plant.

According to certain embodiments, the present invention provides a genetically modified plant having an enhanced expression of at least one of Arabidopsis thaliana 5TPase7 (At5TPase7), Arabidopsis thaliana 5TPase9 (At5TPase9) or homologs thereof, wherein the plant has increased tolerance to environmental abiotic stress compared to a corresponding unmodified plant.

Overexpression of At5TPase7, At5TPase9 or a combination thereof can be obtained by any method as is known to a person skilled in the art. According to certain embodiments, the present invention provides a transgenic plant comprising at least one cell transformed with an isolated polynucleotide encoding at least one of Arabidopsis thaliana 5TPase7 (At5TPase7), Arabidopsis thaliana 5TPase9 (At5TPase9) or a homolog thereof, wherein the plant has increased tolerance to environmental abiotic stress compared to a corresponding non-transgenic plant.

According to some embodiments, the transgenic plant comprises at least one cell transformed with an isolated polynucleotide encoding At5TPase7. According to other embodiments, the transgenic plant comprises at least one cell transformed with an isolated polynucleotide encoding At5TPase9. According to certain additional embodiments, the transgenic plant comprises at least one cell transformed with a combination of the polynucleotide encoding At5TPase7 and the polynucleotide encoding at5TPase9. According to other additional embodiments, the cell is transformed with a single polynucleotide encoding both At5TPase 7 and At5TPase9.

According to certain embodiments, the At5TPase7 comprises the amino acid sequence set forth in SEQ ID NO:1. According to other embodiments, the polynucleotide encoding At5TPase7 comprises the nucleic acid sequence set forth in SEQ ID NO:2. According to further certain embodiments, the At5TPase9 comprises the amino acid sequence set forth in SEQ ID NO:3. According to other embodiments, the polynucleotide encoding the At5TPase9 comprises the nucleic acid sequence set forth in SEQ ID NO:4.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the At5PTase activity of the polypeptide in the context of the present invention, that is to confer tolerance to biotic stress, particularly soil salinity and drought when overexpressed. Specifically, any active fragments of the active polypeptide or protein as well as extensions, conjugates and mixtures are disclosed according to the principles of the present invention.

According to yet other embodiments, the polynucleotide encoding the At5TPase of the present invention are incorporated in a DNA construct enabling their expression in the plant cell. According to some embodiment, the DNA construct comprises at least one expression regulating element selected from the group consisting of a promoter, an enhancer, an origin of replication, a transcription termination sequence, a polyadenylation signal and the like.

According to certain embodiments, the expression of the At5TPase is controlled by its natural promoter. According to other embodiments, the expression of the At5TPase is controlled by a constitutive promoter operable in a plant cell. According to some embodiments, the constitutive promoter is tissue specific.

According to other embodiments, the expression vector further comprises a regulatory element selected from the group consisting of an enhancer, an origin of replication, a transcription termination sequence, a polyadenylation signal and the like.

According to certain embodiments, each of the At5TPase7 and At5TPase9 encoding polynucleotides form part of a separate DNA construct. According to other embodiments the DNA is so designed to express both At5TPase7 and At5TPase9.

According to additional embodiments, the present invention provides a genetically modified plant having inhibited expression of ZEEP1 or a homolog thereof compared to the expression of ZEEP1 or its homolog in a corresponding unmodified plant.

It is to be understood that inhibiting the expression of ZEEP1 may be achieved by various means, all of which are explicitly encompassed within the scope of present invention. According to certain embodiments, inhibiting ZEEP1 expression can be affected at the genomic and/or the transcript level using a variety of molecules that interfere with transcription and/or translation including, but not limited to, antisense, siRNA, Ribozyme or DNAzyme molecule. According to other embodiments, inhibiting ZEEP1 expression is affected by inserting a mutation to the ZEEP1 gene, including deletions, insertions, site specific mutations and the like, as long as the mutation results in down-regulation of the gene expression. According to other embodiments, ZEEP1 expression is inhibited at the protein level using antagonists, enzymes that cleave the polypeptide and the like.

According to certain embodiments, the ZEEP1 protein comprises the amino acid sequence set forth in SEQ ID NO:19. According to other embodiments, the ZEEP1 is encoded by a polynucleotide having the nucleic acid sequence set forth in SEQ ID NO:20.

According to certain embodiments, the genetically modified plant comprises at least one cell comprising a mutated ZEEP1 gene or a homolog thereof, wherein the plant has an increased tolerance to environmental abiotic stress compared to a corresponding unmodified plant.

According to certain typical embodiments, the mutation is within the endonuclease/exonuclease/phosphatase (EEP) domain. According to other embodiments, the genetically modified plant is a transgenic plant comprising at least one cell comprising a ZEEP1 silencing molecule selected from the group consisting of RNA interference molecule and antisense molecule. Each possibility represents a separate embodiment of the present invention.

As used herein, the term “environmental abiotic stress” refers to sub-optimal growth condition or conditions. According to certain embodiments the environmental abiotic stress is selected from the group consisting of soil salinity, drought (water stress), cold stress, heat stress, presence of heavy metals, excess light or a combination thereof. Each possibility represents a separate embodiment of the present invention.

Soil salinity is typically measured as soil electric conductivity (EC). According to certain embodiments, the term “soil salinity” as used herein refers to medium to high soil salinity. Medium soil salinity typically refers to soil electric conductivity of from about 4 deciSiemens per meter (dS/m) to 8 dS/m and high salinity to EC of above 8 dS/m.

Drought (water stress) is typically measured by the water content of the soil. According to certain embodiments, drought conditions refer to soil water content of less than 70%.

According to certain embodiments, the plant is a crop plant selected from the group consisting of plants producing fruit (including vegetables); flower and ornamental plants; grain producing plants crops (wheat, oats, barley, rye, rice, maize); legumes (peanuts, peas soybean lentil etc); forage crops used for hay or pasture; root crops (sweet potatoes etc), fiber crops (cotton, flax etc); trees for wood industry; tuber crops (potato), sugar crops (sugar beet, sugar came) and oil crops (canola, sunflower, sesame etc). Each possibility represents a separate embodiment of the invention.

The present invention also encompasses seeds of the genetically modified plant, wherein plants grown from said seeds comprise at least one cell having an altered expression of at least one EEP encoding gene, and have enhanced tolerance to environmental abiotic stress compared to plants grown from seeds of corresponding unmodified plant.

According to certain embodiments, the plants grown from said seeds comprise at least one cell transformed with a polynucleotide encoding At5TPase selected from the group consisting of At5TPase7 and At5TPase9 or homologs thereof. According to other embodiments, the plants grown from said seeds comprise at least one cell having a mutation in the polynucleotide encoding ZEEP1 or a homolog thereof. According to yet additional embodiments, the plants grown from said seeds comprise at least one cell transformed with a silencing molecule targeted to the polynucleotide encoding ZEEP1 or a homolog thereof. According to certain typical embodiments, the silencing molecule is an RNAi molecule targeted to SEQ ID NO:20.

The present invention further encompasses fruit, leaves or any part of the transgenic plant, as well as tissue cultures derived thereof and plants regenerated therefrom.

According to another aspect, the present invention provides a method for increasing the tolerance of a plant to environmental abiotic stress, comprising (a) transforming a plant cell with at least one isolated polynucleotide encoding At5TPase7, At5TPase9 or a combination thereof; and (b) regenerating the transformed cell into a transgenic plant comprising at least one cell expressing At5TPase7, At5TPase9 or combination thereof having an increased tolerance to environmental abiotic stress compared to a corresponding non-transgenic plant.

According to additional aspect, the present invention provides a method for increasing the tolerance of a plant to environmental abiotic stress, comprising (a) silencing the expression of ZEEP1 gene or a homolog thereof within at least one plant cell; and (b) regenerating the at least one plant cell comprising the silenced gene into a genetically modified plant having an increased tolerance to environmental abiotic stress compared to a corresponding unmodified plant.

The exogenous polynucleotide(s) encoding At5TPase7, At5TPase9 or ZEEP1 silencing molecule according to the teachings of the present invention can be introduced into a DNA construct to include the entire elements necessary for transcription and translation as described above, such that the polypeptides are expressed within the plant cell.

Transformation of plants with an isolated polynucleotide may be performed by various means, as is known to one skilled in the art. Common methods are exemplified by, but are not restricted to, Agrobacterium-mediated transformation, microprojectile bombardment, pollen mediated transfer, plant RNA virus mediated transformation, liposome mediated transformation, direct gene transfer (e.g. by microinjection) and electroporation of compact embryogenic calli. According to one embodiment, the transgenic plants of the present invention are produced using Agrobacterium mediated transformation.

Transgenic plants comprising the polynucleotides of the present invention may be selected employing standard methods of molecular genetics, as are known to a person of ordinary skill in the art. According to certain embodiments, the presence of the transformed polynucleotide is verified by Polymerase Chain Reaction (PCR) using appropriate primers. Alternatively, the DNA construct further comprises a nucleic acid sequence encoding a detection marker enabling a convenient detection of the recombinant polypeptides expressed by the plant cell, for example resistance to antibiotic or a detectable reporter gene. Yet alternatively, the presence of the transformed polynucleotide is verified by planting the plant line overexpressing the polynucleotide encoding at least one At5TPase or homolog thereof and a corresponding control plant having lower expression of said polynucleotide under abiotic stress condition, comparing the growth of the plant line to the growth of the control plant and selecting plant lines having enhanced resistance to the stress condition as compared to said control plant. Similarly, the tolerance to abiotic stress of plants comprising silenced ZEEP1 encoding gene or homolog thereof is compared to the tolerance of a corresponding unmodified plant, and plant lines having enhanced resistance to the stress condition are selected.

Any plant can be transformed with the polynucleotides of the present invention to produce the transgenic plants having enhanced tolerance to soil salinity and/or drought.

According to other aspects the present invention relates to the transgenic plants generated by the methods of the present invention as well as to their seeds, fruit, roots and other organs or isolated parts thereof.

Other objects, features and advantages of the present invention will become clear from the following description and drawings.

### BRIEF DESCRIPTION OF THE DRAWINGS

FIG. 1 shows the results of quantitative RT-PCR analysis of At5TPase2, At5TPase3, At5TPase7 and At5TPase11. Gene expression was measured in A. thaliana roots, taken from control and plant treated with 250 mM NaCl for 6 h. Roots were of mature 4.5 weeks old plants. Inserted square shows expression of At5TPase11 after one week from germination. Transcription was normalized to ACTIN-2 expression. Data shown are the average of three plant replicates.

FIGS. 2A-2E shows the analysis of salt tolerance in Arabidopsis At5ptase mutants. FIG. 2A: Survival of homozygous Arabidopsis At5ptase mutants grown on 150 mM NaCl. Seedlings of At5ptase mutants were grown for 7 d on one-half strength MS medium and then transferred to 150 mM NaCl for 5 days. FIG. 2B-2C: Four-week-old plants grown in soil and irrigated with 200 mM NaCl for 10 days and then with fresh water for 1 week. 2B: wilt type plants; 2C: At5ptase7 mutant plants. FIG. 2D: Mutants of At5ptase7 (Salk_{—}038828 heterozygous, Salk_{—}038842 homozygous, and Salk_{—}040226 homozygous) treated with 200 mM NaCl (top). FIG. 2E: Survival of heterozygous mutant seedlings treated as in FIG. 2A. The T4 heterogeneous Salk mutant lines were selected on kanamycin.

FIGS. 3A-3D shows the salt tolerance of A. thaliana. FIG. 3A: Wild-type and mutant At5ptase1 (SAIL_{—}171A10), At5PTase2 (SAIL_{—}138G01) and At5ptase9 (salk_{—}090899) plants germinated on one-half-strength MS (½ MS) supplemented with 150 mM NaCl. Plants with true leaves that were predominately green were scored after 10 days. FIG. 3B: Expression of the At5PTase9 gene in wild-type (wt) and mutant plants (salk_{—}090899, salk_{—}090905). FIG. 3C: Wild-type and At5ptase9 mutants germinated on ½ MS, and transferred to ½ MS supplemented with 150 mM NaCl or to ½ MS (control, side pictures) after 7 days. Pictures were taken after 10 days. FIG. 3D: Wild-type A. thaliana plants transformed with At5PTase9 gene driven by the CaMV promoter. The transgenic plants (OE) were germinated and grown on 150 mM NaCl. Pictures were taken after 10 days.

FIG. 4 shows the growth of wild-type and At5ptase9 mutants in 150 mM NaCl. Wild-type and At5ptase9 mutants (Salk_{—}090905) were germinated on ½ MS and after seven days transferred to plates supplemented with 150 mM NaCl or to ½ MS (control).

FIGS. 5A-5B demonstrates the salt tolerance of transgenic plants overexpressing the At5PTase7 gene. FIG. 5A: Expression of the At5PTase7 gene in transgenic plants (OE, overexpressors); WT, Wild type. FIG. 5B: Wild-type (left) and At5PTase7-overexpressing plants (right). Transgenic At5PTase7 plants were germinated and grown on plates supplemented with 150 mM NaCl for 1 week. There was no difference in the appearance and growth of the transgenic plants as compared with the wild-type plants when not stressed with NaCl.

FIGS. 6A-6B demonstrates the drought tolerance of transgenic plants overexpressing the At5PTase9 (At2g01900) gene. Wild-type, At5ptase9 mutant and At5ptase9-overexpressing plants were grown in pots filled with soil under standard growth conditions (16 hours daylight, 24° C., watered two times a week) for 6 weeks, after which irrigation was stopped (FIG. 6A). Pictures were taken after 2 weeks without water. The watered control plants are shown in FIG. 6B.

FIGS. 7A-7C shows subcellular localization of At5PTase7. FIG. 7A: Analysis of stably transformed Arabidopsis plants with At5PTase7-GFP. Localization of At5PTase7-GFP was in the nucleus of root hair cells. Nuclei of 7-day-old transgenic seedlings were stained for 10 min with DAPI fluorescent dye. Image analysis was done using the Olympus IX70 epifluorescence microscope (1003 oil-immersion objective) equipped with a Coolpix 950 camera (Nikon). GFP speckles can be seen in the nucleus. BF, Bright field. Bar=5 μm. FIG. 7B: Localization of At5PTase7-GFP in Arabidopsis epidermal leaf cells. Image analysis was done using a Zeiss LSM 510 Laser Scanning Microscope with a 633 oil-immersion objective. The inset shows GFP expressed from the 35S promoter in epidermal leaf cells (control). Bar=5 μm. FIG. 7C: Localization of At5PTase7-GFP in the root cells of Arabidopsis. Seven-day-old transformed seedlings were incubated for 10 min with DAPI fluorescent stain, which stains the nucleus. Image analysis was done using the Olympus IX70 epifluorescence microscope (403 oil-immersion objective) equipped with a Coolpix 950 camera (Nikon). The insets show the magnification of one representative root cell nucleus. Speckles of GFP can be seen in the nucleus. Bar=10 μm.

FIGS. 8A-8B Shows analysis of tissue specific expression of the At5TPase9 gene in wild-type A. thaliana. FIG. 8A: At5TPase9 gene expression in shoots. FIG. 8B: At5TPase9 gene expression in roots. A. thaliana was transformed with the At5PTase9 including its native promoter (˜1000 bp upstream of ATG) fused to GUS. One week-old plants were stained for X-GlucA and visualized with Olympus IX70 microscope (top) or directly photographed in the Petri dish with a Canon digital camera.

FIGS. 9A-9C shows the salt-induced Reactive Oxygen Species (ROS) production in wild-type and At5ptase mutant plants. FIG. 9A: Arabidopsis seedlings were grown on agar plates supplemented with ½ MS nutrient medium. After 7 days, the seedlings were removed from the agar plates, washed, and transferred into medium with 200 mM NaCl. ROS generation was measured in the root tip and transition zone by confocal microscopy with 2′,7′-dichlorodihydrofluorescein diacetate (H₂DCFDA) in At5Ptase3, At5Ptase7, and At5Ptase11 roots at 15 min after stimulation with 200 mM NaCl. wt, Wild type. FIG. 9B: Representative confocal images of wild-type and At5ptase7 mutant plants treated with ½ MS or 200 mM NaCl (magnification, 3200). Seedlings were grown and treated as in FIG. 9A. Bars=50 μm. FIG. 9C: Restoration of ROS production in the At5ptase7 mutants during salt stress. Seedlings were grown and treated as in FIG. 9A. The exogenous PtdIns-phosphates (indicated) were incubated with the seedlings for 1 h before treatment with NaCl. RU, Relative units.

FIG. 10 shows the production of reactive oxygen species in A. thaliana roots during salt stress. ROS production in the indicated mutant Arabidopsis lines was measured after exposure to 0.2 M NaCl for 10 min. Observation was done in four days-old seedlings stained with H₂DCFDA by epi-fluorescent microscopy using narrow-band GFP filter (Ex 485±10 nm/Em 525±10 nm). ROS were quantified by ImagePro Plus analysis package in root tip and elongation zones. Bar=60 μm. Error bars indicate standard deviation of the mean (N=12). Presented above the bars are representative images of ROS production in the roots.

FIGS. 11A-11B shows subcellular localization of salt-induced ROS production. FIG. 11A: Measurements of ROS production localized to the nucleus in the root tip zone of wild-type and At5ptase7 seedlings after 20 min of stimulation with 200 mM NaCl. The seedlings were treated as in FIG. 9A. Shown are averages of 10 nuclei measurements in five plant replicates. RU, Relative units. FIG. 11B: Representative images of wild-type and At5ptase7 root tips. High-magnification images of ROS accumulation in the cytoplasm and the nucleus of root tip cells by confocal microscopy (magnification, ×630) are shown. The seedlings were treated as in FIG. 9A for 20 min with 200 mM NaCl with the supplement of the DAPI fluorescent stain. Shown is a representative layer of a 1-mm optical section along the z axis. Insets show a higher magnification of one representative cell. Bar=10 μm.

FIG. 12 represents influx of free cytosolic Ca²⁺_{cyt} in A. thaliana roots during salt stress. Four days-old Arabidopsis seedlings were loaded with 10 μM Fluo4-AM in the presence of pluronic acid for 120 min. and treated with 0.2 M NaCl. The cytosolic Ca²⁺ concentration was assayed 10 min. after the salt treatment by epi-fluorescent microscopy using narrow-band GFP filter. The root zones included the root tip, transition and the lower half of the elongation. [Ca²⁺c_{yt}] was quantified with ImagePro Plus analysis package. The y axis represents the intensity measured in arbitrary units. Error bars indicate standard deviation of the mean (N=12).

Inset: Wild-type A. thaliana pretreated or not treated (Cont) with 18 μM diphenylene iodonium (DPI) for 60 min., followed by addition of 0.2 M NaCl for 10 min.

FIG. 13 shows the Bblk-flow endocytosis as measured by internalization of the membrane dye FM4-64. Wild-type and mutant seedlings were stained with the lipophilic styryl membrane tracker probe, FM4-64. The FM4-64 was visualized by epi-fluorescent microscopy. Pictures were taken 10 min. after dye addition. Inhibitor of endocytosis, Tyrphostin A23, was added 60 min. prior to addition of FM4-64. FM4-64 uptake was quantified with ImagePro Plus analysis package. Error bars indicate standard deviation of the mean (N=10). The y axis represents the intensity measured in arbitrary units.

FIGS. 14A-14B shows endocytosis-dependent ROS production and Ca2⁺_{cyt} influx. FIG. 14A: ROS production: Arabidopsis seedlings were pre-treated with 175 μM Tyrphostin A23 (TyrA23) or 20 μM PAO for 60 min., then stained with 10 μM H₂DCFDA and immediately stimulated with 0.2 M NaCl for 10 min. ROS production was quantified using ImagePro Plus software as described in FIG. 12. The y axis represents the intensity measured in arbitrary units. Error bars indicate standard deviation of the mean (N=12). Above, representative pictures of ROS production in the roots. Bar=60 μm. FIG. 14B: Ca²⁺ influx: four days-old Arabidopsis seedlings were loaded with 10 μM Fluo4-AM in the presence of pluronic acid for 120 min. After 60 min., endocytosis inhibitors, TyrA23 or PAO, were added for 60 min., and the plants stimulated with 0.2 M NaCl (for 10 min) as described in FIG. 14A. Ca²⁺ influx was assayed by epi-fluorescent microscopy using narrow-band GFP filter (Ex 485±10 nm/Em 525±10 nm). ROS were quantified using ImagePro Plus software. Error bars indicate standard deviation of the mean (N=12). Representative pictures show ROS production in the roots. Bar=60 μm.

FIGS. 15A-15B demonstrates that inhibition of endocytosis decreases plant salt tolerance. Seeds of wild-type plants were sown on agar plates containing ½×MS supplemented with 20 mM PAO and/or 120 mM NaCl. FIG. 15A: Pictures taken 6 days after germination. FIG. 15B: Percentage of germination and sprout survival (out of the total sown).

FIGS. 16A-16B shows the substrate specificity of At5PTase3 and At5PTase7. FIG. 16A: Activity of At5PTase using fluorescent PtdIns(4,5)P₂ substrate with immunoprecipitated 5PTases. The reaction mixtures containing 1.5 μg of the fluorescent PtdIns(4,5)P₂ substrate were incubated for 1 h at room temperature. Lane 1, PtdIns(4)P substrate incubated with reaction buffer only; lane 2, PtdIns(4,5)P₂ substrate incubated with reaction buffer only. Other lanes contain reaction mixtures with the following immunoprecipitants (IPs): lane 3, At5PTase3-IP; lane 4, At5PTase7-IP; lane 5, control IP from mock-transfected S2 cells. FIG. 16B: Activity of At5PTase using fluorescent PtdIns(3,4,5)P₃ substrate with immunoprecipitated 5PTases. Phosphatase reactions (lanes 3 and 4) containing 1.5 μg of the fluorescent PtdIns(3,4,5)P₃ substrate were incubated for 1 h at room temperature. Lane 1, PtdIns(3,4)P₂substrate incubated with reaction buffer only; lane 2, PtdIns(3,4,5)P₃ substrate incubated with reaction buffer only. Other lanes contain reactions with the following IPs: lane 3, At5PTase3-IP; lane 4, At5PTase7-IP; lane 5, control IP from mock-transfected S2 cells. The migration of standards is indicated.

FIGS. 17A-17B demonstrates the expression of stress-responsive genes in wild-type, At5ptase9 mutants and At5PTase9 overexpressing plants following salt treatment. FIG. 17A: expression of RD29A, RD22, AtCBF2, AtRab18C and AtICE1 genes in the roots was analyzed by semi-quantitative RT-PCR. Wild-type and At5ptase9 mutants were germinated on agar plates, grown for two weeks and then treated with 0.2 M NaCl, for 3 hours. RNA was extracted from the plant roots using the Tri-Reagent Kit (Molecular Research Center Inc.) and cDNA was prepared by reverse transcriptase SuperScript III kit (Invitrogen, Carlsbad, Calif.). FIG. 17B: the effect of endocytosis inhibitor on induction of stress-responsive genes during salt stress. Plants were treated with 200 mM NaCl with or without 20 μM Phenylarsine oxide (PAO) for 3 hours prior to extraction of RNA.

FIG. 18 shows the increased tolerance of At1g11800 mutant (designated herein ZEEP1) to salt stress. Seeds (wilt type and mutants) were germinated on ½ strength MS medium (without sucrose) supplemented with 150 mM NaCl.

FIG. 19 shows the structure of the ZEEP1 gene (At1g11800). T-DNA insertions, as produced by the Salk Institute, CA USA are indicated. Note that the gene direction in the Figure is from right to left.

FIG. 20 shows the slat tolerance of two ZEEP1 mutants, zeep1-1 and zeep1-2. Seeds of wild type, zeep1-1 mutant and zeep1-2 mutant were germinated in ½ strength MS medium and transferred to saline condition. Plant survival was measured after 7 days.

FIGS. 21A-21B demonstrates the expression pattern of ZEEP1 under various environmental conditions. FIG. 21A: analysis of ZEEP1 expression in seedlings using the data from Bio-Array Resource for Arabidopsis Functional Genomics site (http://bbc.botany.utoronto.ca). FIG. 21B: Analysis of ZEEP1 expression during salt (200 mM NaCl) stress in mature plants showing that it maintained the down-regulation.

FIGS. 22A-22B shows ROS production in root tip and transition zone of wild-type and zeep1 mutants (zeep1-1 and zeep1-2) 30 min. after stimulation with 200 mM NaCl. ROS generation was by confocal microscopy with H₂DCFDA. FIG. 22A: ROS production in relative units. FIG. 22B: Representative pictures of confocal images of wild type and mutants treated with H₂O or 200 mM NaCl.

FIG. 23 shows draught tolerance of zeep1 mutants compared to wild type. Seedlings of zeep1-1 and zeep1-2 mutants were transferred to soil after seven days in Petri plates and grown in constant watering regime under long-conditions. Irrigation was stopped after 5 weeks and the plant left to wilt. When the plants began to show signs of turgor loss they were re-watered.

FIGS. 24A-24C demonstrates the expression of stress-responsive genes in wild-type, zeep1 mutants and zeep1 overexpressing plants following salt treatment. Conditions are as described for FIG. 17.

### DETAILED DESCRIPTION OF THE INVENTION

The present invention answers the need for means and methods for producing plants that are tolerant to sub-optimal growth conditions, particularly plant tolerant to soil salinity and/or low water availability.

Unexpectedly, the present invention now shows that plants overexpressing particular phosphatidylinositol 5-phosphatases (5PTases) show enhanced tolerance to salt and/or water stress as well as to other abiotic stresses compared to corresponding wild type plants. The present invention further shows that plants harboring a mutation in the EEP protein At1g11800 also show enhanced tolerance abiotic stresses. The transgenic plants of the present invention show tolerance to highly saline growth medium or irrigating water (150 mM NaCl and above).

Definitions

The term “plant” is used herein in its broadest sense. It includes, but is not limited to, any species of woody, herbaceous, perennial or annual plant. It also refers to a plurality of plant cells that are largely differentiated into a structure that is present at a stage of the plant development capable of producing crop.

As used herein, the term “crop plant” refers to a plant with at least one part having commercial value. The term encompasses plants producing edible fruit (including vegetables), plants producing grains (as a food, feed and for oil production), plant producing flowers and ornamental plants, legumes, root crops, tuber crops, leafy crops and the like.

The term “Endonuclease/Exonuclease/Phosphatase” or “EEP”, used herein interchangeably refer to a conserved structural domain present in many proteins, referred to herein as “Endonuclease/Exonuclease/Phosphatase protein family” or “EEP protein family”. The diverse proteins of this family share a common catalytic mechanism of cleaving phosphodiester bonds. Their substrates range from nucleic acids to phospholipids and perhaps, proteins.

The term “At5TPase” refers to Arabidopsis thaliana phosphatidylinositol 5-phosphatase. At5TPase7 is denoted by accession No. At2g32010 having the amino acid sequence set forth in SEQ ID NO:1. According to certain embodiments, the At5TPase7 is encoded by the polynucleotide having SEQ ID NO:2. At5TPase9 is denoted by accession No. At2g01900 having the amino acid sequence set forth in SEQ ID NO:3. According to certain embodiments, the At5TPase7 is encoded by the polynucleotide having SEQ ID NO:4.

The term “ZEEP1” refers to Arabidopsis thaliana EEP protein denoted by accession No. At1g11800 having the amino acid sequence set forth in SEQ ID NO:19. According to certain embodiments, the ZEEP1 protein is encoded by the polynucleotide having SEQ ID NO:20.

The term “environmental abiotic stress condition(s)” as used herein refers to sub-optimal growing condition(s). These include water stress (drought); high soil salinity; extreme temperature (cold stress—temperatures below the optimal for growth and heat stress—temperatures above the optimal for growth of a certain plant species); presence of heavy metals, particularly in the soil, at concentrations deleterious to the growth of a certain plant species; and light intensity over the optimal for growth of the plant species. As used herein, the term “soil salinity” refers to the salt concentration of the soil solution in terms of g/I or electric conductivity (EC) in dS/m. EC of 5 is about 60 mM NaCl; EC of 10 is about 120 mM NaCl and of EC 12.5 is about 250 mM NaCl. Sea water may have a salt concentration of 30 g/l (3%) and an EC of 50 dS/m. Soils are considered saline when the EC>4. When 4<EC<8, the soil is called moderately saline and when 8<EC the soil is called highly saline.

The terms “water stress”, “drought conditions” and “low soil water content” are used herein interchangeably and refer to sub-optimal soil hydration conditions for the growth of a particular plant species. Soil hydration can be measured by various methods as is known to a person skilled in the art, depending on the soil type. According to certain embodiments, the soil water content is measured relative to the maximum amount of water that a given soil can retain (“field capacity”) as weight/weight percentage. According to these embodiments, drought conditions refer to soil water content of less than 70%.

It is to be understood that different plant species show different response to a certain abiotic stress, particularly to soil salinity and soil water content. Accordingly, as used herein the terms “a plant having an enhanced tolerance” or “a plant having an enhanced resistance” refer to at least about 2%, at least about 3%, at least about 4%, at least about 5%, at least about 10%, at least about 1%, at least about 20%, at least about 30%, at least about 40%, at least about 50%, at least about 6%, at least about 70%, or at least about 80% and more increase in the plant abiotic stress tolerance as measured by at least one of growth, biomass, yield, fertilizer use efficiency and water use efficiency of the transgenic plant of the invention (i.e. a plant transformed with at least one polynucleotide of the invention) compared to a corresponding non-transgenic plant of the same species, wherein both plants are grown under the same normal or stress conditions.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, “nucleic acid sequence”, and “isolated polynucleotide” are used interchangeably herein. These terms encompass isolated nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

Typically, the term RNAi molecule refers to single- or double-stranded RNA molecules comprising both a sense and antisense sequence. For example the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

According to some embodiments of the invention, the isolated polynucleotide of the invention encodes a polypeptide having an amino acid sequence at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more say 100% homologous to the amino acid sequence set forth in any one of SEQ ID NOs: 1,3 and 19.

The term “homology”, as used herein, refers to a degree of sequence similarity in terms of shared amino acid or nucleotide sequences. There may be partial homology or complete homology (i.e., identity). For amino acid sequence homology amino acid similarity matrices (e.g. BLOSUM62, PAM70) may be utilized in different bioinformatics programs (e.g. BLAST, FASTA, MPsrch or Scanps) and may employ the use of the Smith and Waterman algorithms, and/or global/local or BLOCKS alignments for analysis, for example. Different results may be obtained when performing a particular search with a different matrix or with a different program. Degrees of homology for nucleotide sequences are based upon identity matches with penalties made for gaps or insertions required to optimize the alignment, as is well known in the art.

According to certain embodiments, the homologous polynucleotide is an ortholog. The term “ortholog” as used herein refers to homologous genes found in two or more species that can be traced to a common ancestor.

The term “DNA construct” as used herein refers to an artificially assembled or isolated nucleic acid molecule which includes the gene or polynucleotide of interest. The construct may further include a marker gene which in some cases can also be the gene of interest. In certain embodiments, the DNA construct is an expression vector further comprising appropriate regulatory sequences, operably linked to the gene of interest. It should be appreciated that the inclusion of regulatory sequences in a construct is optional, for example, such sequences may not be required in situations where the regulatory sequences of a host cell are to be used. The term construct includes vectors (including expression vectors and transformation vectors) but should not be seen as being limited thereto. According to certain typical embodiments, the DNA construct of the present invention is an expression vector.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. According to typical embodiments of the present invention, the polynucleotide encoding At5TPase is operably linked to the regulatory sequences in a sense orientation.

The terms “promoter element,” “promoter,” or “promoter sequence” as used interchangeably herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. New promoters of various types useful in plant cells are constantly being discovered; numerous examples may be found in Okamuro J K and Goldberg R B (1989) Biochemistry of Plants 15:1-82). According to certain typical embodiments, the DNA construct of the present invention comprises a constitutive promoter.

As used herein, the term an “enhancer” refers to a DNA sequence which can stimulate promoter activity, and may be an innate element of the promoter or a heterologous element inserted to enhance the level or tissue-specificity of a promoter.

The term “expression”, as used herein, refers to the production of a functional end-product e.g., an mRNA or a protein.

The term “genetically modified plant” refers to a plant comprising at least one cell genetically modified by man. The genetic modification includes modification of an endogenous gene(s), for example by introducing mutation(s) deletions, insertions, transposable element(s) and the like into an endogenous polynucleotide or gene of interest. Additionally or alternatively, the genetic modification includes transforming the plant cell with heterologous polynucleotide. A “genetically modified plant” and a “corresponding unmodified plant” as used herein refer to a plant comprising at least one genetically modified cell and to a plant of the same type lacking said modification, respectively.

The term “transgenic” when used in reference to a plant or seed (i.e., a “transgenic plant” or a “transgenic seed”) refers to a plant or seed that contains at least one heterologous transcribeable polynucleotide in one or more of its cells. According to the teachings of the present invention, the transgenic plant comprises at least one polynucleotide encoding At5TPase or a homolog thereof, and/or a silencing polynucleotide targeted to the ZEEP1 gene or a homolog thereof. The terms “transgenic plant material” and a “corresponding non transgenic organism” refer broadly to a plant, a plant structure, a plant tissue, a plant seed or a plant cell that contains at least one heterologous gene of the invention and to a plant, plant structure, plant tissue, plant seed or plant cell of the same type lacking said heterologous transcribable polynucleotide, respectively.

The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of the protein (e.g. -glucuronidase) encoded by the exogenous polynucleotide.

The term “transient transformant” refers to a cell which has transiently incorporated one or more exogenous polynucleotides. In contrast, the term “stable transformation” or “stably transformed” refers to the introduction and integration of one or more exogenous polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA. It is to be understood that a plant or a plant cell transformed with the nucleic acids, constructs and/or vectors of the present invention can be transiently as well as stably transformed.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

### PREFERRED MODES FOR CARRYING OUT THE INVENTION

The present invention provides genetically modified plants having altered expression of at least one Endonuclease/Exonuclease/Phosphatase (EEP) protein that show enhanced tolerance to abiotic stresses compared to corresponding unmodified plants.

The EEP genes are found in a large number of proteins, including magnesium dependent endonucleases and phosphatases. There are more than 100 EEP genes in the Arabidopsis thaliana genome (http://metnetonline.org). These genes were shown to be involved in intracellular signaling, actin cytoskeleton organization and regulation of endocytosis. EEPs have been shown to be involved in regulation of plant growth and development and in plant responses to environmental stresses. Some members of the EEPs can also bind calcium ions, thus expanding their regulatory functioning.

According to one aspect, the present invention provides a genetically modified plant comprising at least one cell having altered expression of at least one of Endonuclease/Exonuclease/Phosphatase (EEP) protein corresponding to Arabidopsis thaliana 5TPase7 (At5TPase7), Arabidopsis thaliana 5TPase9 (At5TPase9), Arabidopsis thaliana ZEEP1 or homologs thereof wherein the plant has increased tolerance to environmental abiotic stress compared to a corresponding unmodified plant.

According to certain embodiments, the present invention provides a genetically modified plant having an enhanced expression of at least one of Arabidopsis thaliana 5TPase7 (At5TPase7), Arabidopsis thaliana 5TPase9 (At5TPase9) or homologs thereof, wherein the plant has increased tolerance to environmental abiotic stress compared to a corresponding unmodified plant.

According to certain embodiments, the transgenic plant comprises at least one cell transformed with a combination of the polynucleotide encoding At5TPase7 and the polynucleotide encoding at5TPase9.

According to certain embodiments, the At5TPase7 comprises the amino acid sequence set forth in SEQ ID NO:1. According to other embodiments, the polynucleotide encoding At5TPase comprises the nucleic acid sequence set forth in SEQ ID NO:2. According to further certain embodiments, the At5TPase9 comprises the amino acid sequence set forth in SEQ ID NO:3. According to other embodiments, the polynucleotide encoding the At5TPase9 comprises the nucleic acid sequence set forth in SEQ ID NO:4.

Phosphatidylinositides (PtdIns) were described in plants already about 50 years ago; however, their involvement in different metabolic processes became recognized only recently (Ercetin and Gillaspy, 2004, ibid). As of today, PtdIns are thought to function in intracellular vesicle trafficking and cytoskeleton organization. PtdIns signaling is mediated by differential phosphorylation of the inositol ring, which is carried out by specific kinases and phosphatases. The plant genomes possess more genes encoding 5PTases than yeast or animals. The first mutational analysis of Phosphatidylinositol 5-Phosphatase genes was performed in Saccharomyces cerevisiae. Since the mutants could grow on basal medium, the authors concluded that these enzymes were not essential for yeast development (Stolz L E et al., 1998. Genetics 148, 1715-1729). Nevertheless, compromised growth in standard medium was observed in some of the double mutants, suggesting some essential function of 5PTases. Similarly, to date most of At5ptase mutants that were analyzed in A. thaliana (altogether 10 out of 15) appeared normal when grown under standard conditions.

The present invention shows for the first time that plant harboring a mutant At5PTase7 gene, and even more significantly plants harboring a mutant At5PTase9 gene are highly sensitive to saline conditions (150-200 mM NaCl). Such salt sensitivity was not observed in plants harboring other 5TPase mutants, indicating a unique role of At5TPase7 and At5TPase9 in the plant abiotic stress response.

According to additional embodiments, the present invention provides a genetically modified plant having inhibited expression of ZEEP1 or a homolog thereof compared to the expression of ZEEP1 or its homolog in a corresponding unmodified plant.

Unexpectedly, it was found that a plant harboring a mutation in an EEP encoding gene, designated ZEEP1 (the mutated gene designated zeep1) is highly tolerated to salt stress. Analysis of the ZEEP1 encoded protein showed that in addition to the Endonuclease/exonuclease/phosphatase (EEP) motif, this protein further contains Zinc Finger motif. Accordingly, the protein, denoted by accession No. At1g11800 was designated ZEEP1 (Zinc Finger EEP).

Zinc finger motif is a common protein domain present in all eukaryotes (Ciftci-Yilmaz S and Mittler R. 2008. Cellular and Molecular Life Sciences 65(7-8), 1150-1160). Just in Arabidopsis thaliana genome there are more than 1000 genes that code for Zinc finger proteins. The Zinc finger proteins have been implicated in plants in many processes associated with development and physiological adaptation responses, including stress signaling (for example, Sakamoto Set al. 2004. The Plant Journal 60(4), 744-754.; Kim S et al. 2006. Plant Cell 18(11): 2985-2998). The present invention further shows that a mutation within the EEP domain of ZEEP1 results in a better tolerance to abiotic stress compared to a mutation within the Zinc Finger motif (FIG. 19).

The major processes involved in salt stress response were further analyzed in the transgenic plant having mutated At5PTase7 or At5PTase9 gene and in plants overexpressing these genes, as well as in plants harboring a mutated zeep1 gene.

The development of oxidative stress is an established consequence of salt stress (Smirnoff, 1998, ibid). It was also shown that ROS production during salt stress is caused by NADPH oxidase (Mazel A et al., 2004. Plant Physiol. 134, 118-128). Normal ROS production was measured in At5TPase11 and At5TPase1 plant, and slightly lower ROS levels were observed in root tips but not in the elongation zone of At5TPase3 mutant plants. However, significantly lower ROS production was observed in At5TPase7 and At5TPase9 mutant plants. In zeep1 mutant plants, salt treatment caused only minor ROS production in zeep1-1 mutants (having the mutation in the EEP domain) and slightly higher ROS production ins zeep1-2 mutants (having the mutation in the Zinc Finger motif); a sharp increase in ROS accumulation was shown in the control plants harboring the wild type ZEEP1 gene. Without wishing to be bound by any specific theory or mechanism of action, these results show that increased tolerance to abiotic stress, particularly slat stress of the genetically modified plants of the present invention i8s associated with reduced production of reactive oxygen species.

The influx of cations from the apoplast into cytosol is thought to occur by ion channels, and multiple general and specific types of channels located in the plasma membrane and in organellar membranes have been identified in plant cells. Recently, a direct uptake of Ca²⁺ from the external medium into endosomes was described (Menteyne A et al., 2006. Current Biology 16, 1931-1937). In that mechanism, vesicles produced by endocytosis become first attached to plasma membrane that are later released into the cytosol. Calcium release also occurs from intracellular stores, which are regulated by Ins(1,4,5)P₃ produced by phospholipase C (Munnik and Vermeer, 2010).

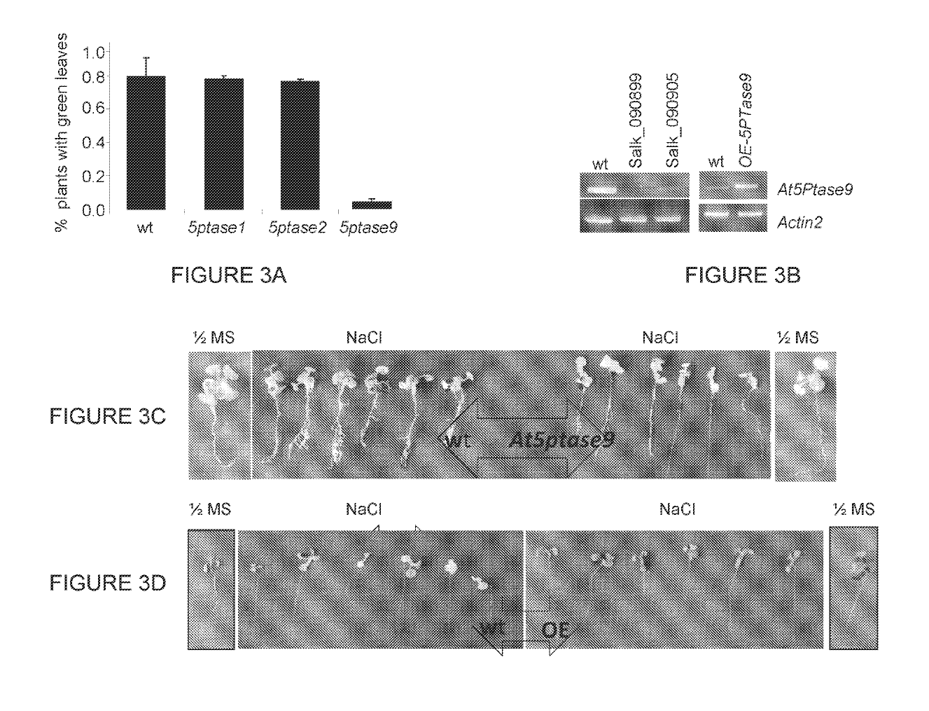

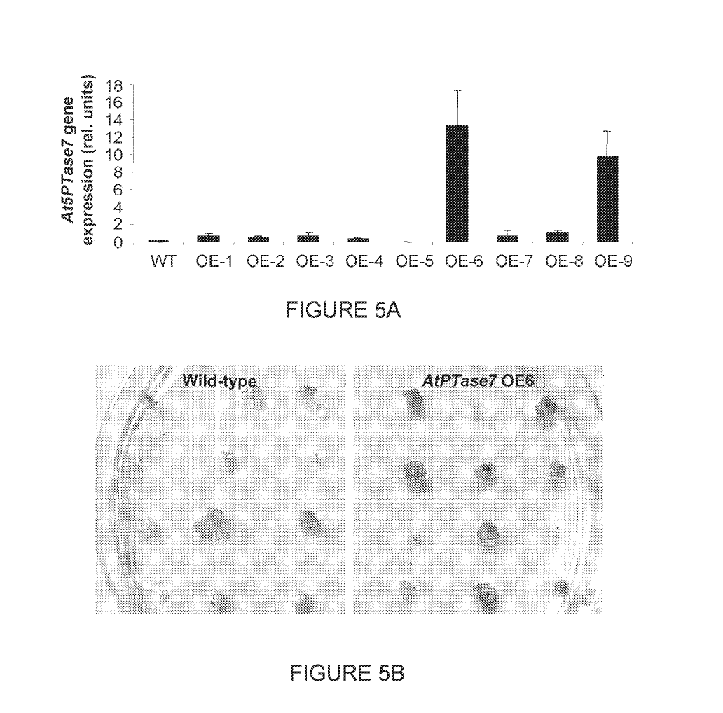

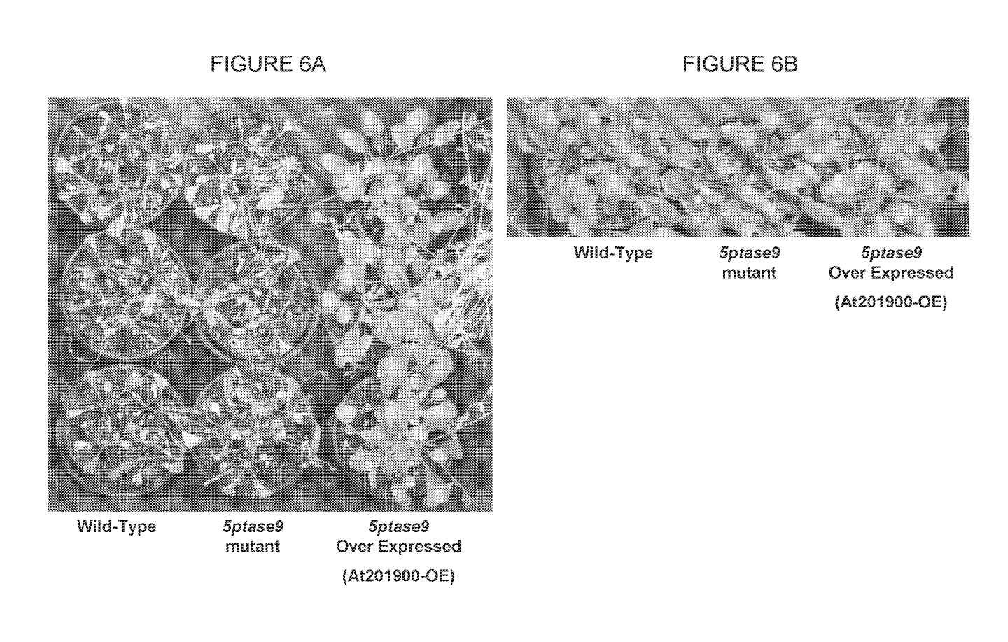

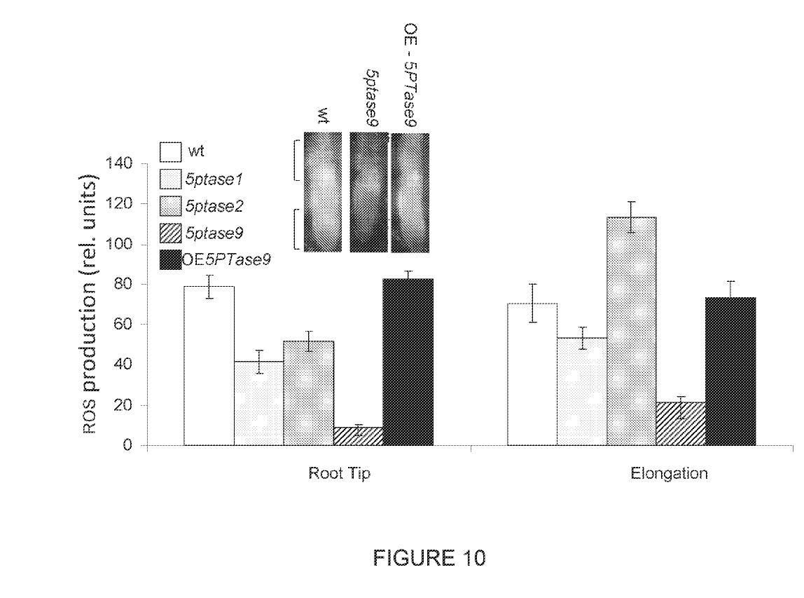

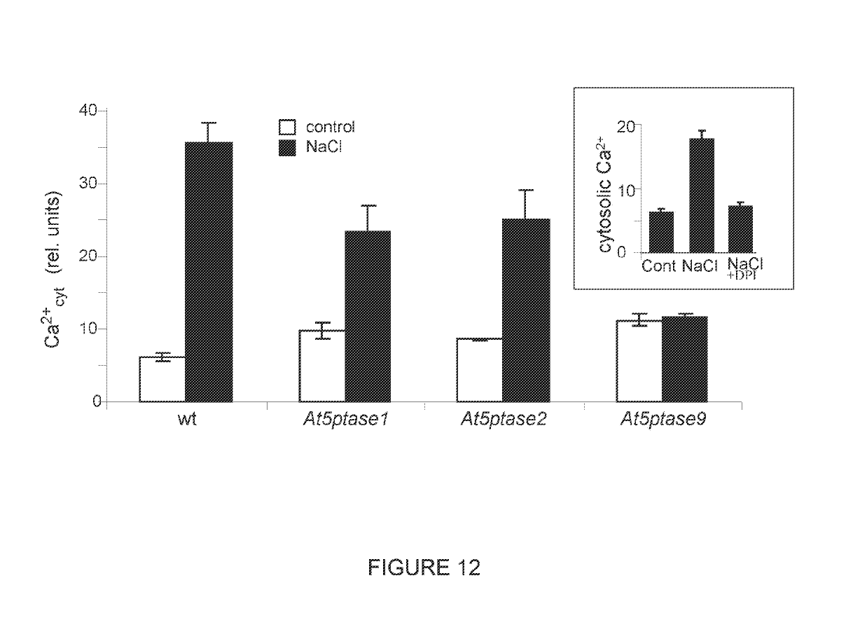

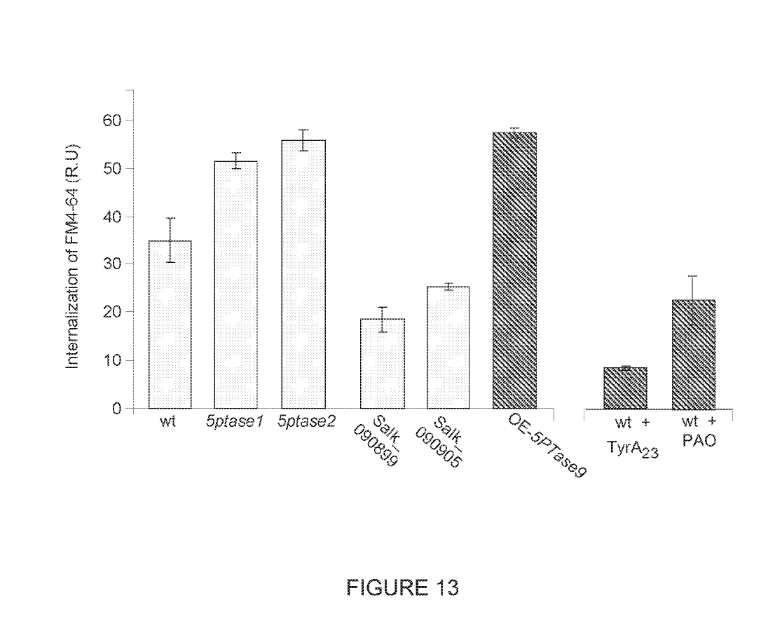

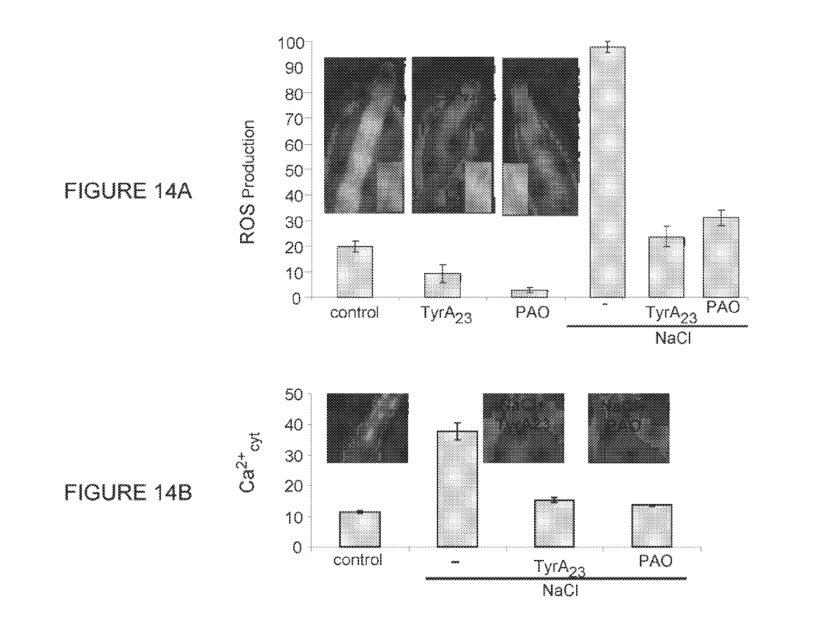

It has been shown previously that salt stress induced endocytosis (Leshem et al., 2007, ibid). PtdIns(4,5)P₂ was shown to accumulate in plants during salt-stress, causing Ca²⁺ mobilization (DeWald D B et al., 2001. Plant Physiol 126, 759-769.), and the association of PtdIns(4,5)P₂ with clathrin-coated vesicles plays a major role in endocytosis. The present invention now shows that endocytosis coincides with Ca²⁺_{cyt} influx (FIG. 12) and intracellular generation of ROS (FIG. 10). Moreover, inhibition of endocytosis blocked both, ROS production and Ca²⁺_{cyt} influx (FIGS. 14A and B, respectively). In mutant At5TPase19 plants, endocytosis was significantly blocked, while in plants overexpressing At5TPase19 ROS production was reestablished (FIG. 10) and endocytosis (FIG. 13). Furthermore, the present invention shows for the first time that transgenic plants overexpressing the At5TPase7 gene (FIG. 5) and the At5TPase9 gene (FIG. 3D) showed improved tolerance to salt stress. Transgenic plants overexpressing the At5TPase9 gene were also highly tolerant to drought stress (FIG. 6).

In addition, At5ptase9 mutants failed to induce stress-responsive genes: AtRD22, RD29A and AtRab18C. Inhibitors of endocytosis prevented induction of RD29A gene in wild-type plants. In summary, we show that At5PTase9 regulates plant drought and salt tolerance, by coordinating endocytosis, ROS production, Ca²⁺ influx and Na⁺ compartmentation, as well as induction of stress-responsive genes.

According to another aspect, the present invention provides a method for increasing the tolerance of a plant to environmental abiotic stress, comprising (a) transforming a plant cell with at least one isolated polynucleotide encoding At5TPase7, At5TPase9 or a combination thereof; and (b) regenerating the transformed cell into a transgenic plant comprising at least one cell expressing At5TPase7, At5TPase9 or combination thereof having an increased tolerance to environmental abiotic stress compared to a corresponding non-transgenic plant.

According to certain embodiments, the isolated polynucleotide encoding At5TPase comprises the nucleic acid sequence set forth in SEQ ID NO:2. According to other embodiments, the isolated polynucleotide encoding the At5TPase9 comprises the nucleic acid sequence set forth in SEQ ID NO:4.

According to additional aspect, the present invention provides a method for increasing the tolerance of a plant to environmental abiotic stress, comprising (a) silencing the expression of ZEEP1 gene or a homolog thereof within at least one cell of the plant; and (b) regenerating the at least one cell comprising the silenced gene into a genetically modified plant having an increased tolerance to environmental abiotic stress compared to a corresponding unmodified plant.

According to certain embodiments, the abiotic stress condition is selected from the group consisting of water stress (drought), high soil salinity, extreme temperatures, presence of heavy metals or high light intensity.

According to certain typical embodiments, the abiotic stress is water stress (drought). According to other typical embodiments the abiotic stress is high soil salinity.

Methods for transforming a plant cell with nucleic acids sequences according to the present invention are known in the art. As used herein the term “transformation” or “transforming” describes a process by which a foreign DNA, such as a DNA construct, including expression vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into a plant cell.

There are various methods of introducing foreign genes into both monocotyledonous and dicotyledonous plants (Potrykus I 1991. Annu Rev Plant Physiol Plant Mol Biol 42, 205-225; Shimamoto K. et al., 1989. Nature 338, 274-276).

The principal methods of the stable integration of exogenous DNA into plant genomic DNA includes two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). The floral dip transformation method is typically used to transform the model plant Arabidopsis (Clough S J and Bent A F, 1998. Plant J 16:735-743). A supplementary approach employs the Agrobacterium delivery system in combination with vacuum infiltration. The Agrobacterium system is especially useful in the generation of transgenic dicotyledonous plants.

Direct DNA uptake: There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

Inhibiting the expression of ZEEP1 can be affected by any method as is known to a person skilled in the art. According to certain embodiments, inhibition is affected at the genomic and/or the transcript level by insertion of mutation or by transforming the plant with a silencing molecule, including antisense and RNAi molecule(s). According to other embodiments, ZEEP1 expression is inhibited at the protein level using antagonists, enzymes that cleave the polypeptide and the like.

Mutations can be introduced into the ZEEP1 gene using, for example, site-directed mutagenesis (see, e.g. Wu Ed., 1993 Meth. In Enzymol. Vol. 217, San Diego: Academic Press; Higuchi, “Recombinant PCR” in Innis et al. Eds., 1990 PCR Protocols, San Diego: Academic Press, Inc). Such mutagenesis can be used to introduce a specific, desired amino acid insertion, deletion or substitution. Chemical mutagenesis using an agent such as Ethyl Methyl Sulfonate (EMS) can be employed to obtain a population of point mutations and screen for mutants of the ZEEP1 genes that may become silent or down-regulated. Methods relaying on introgression of genes from natural populations can be also used. Cultured and wild types species are crossed repetitively such that a plant comprising a given segment of the wild genome is isolated. Certain plant species, for example Maize (corn) or snapdragon have natural transposons. These transposons are either autonomous, i.e. the transposas is located within the transposon sequence or non-autonomous, without a transposas. A skilled person can cause transposons to “jump” and create mutations. Alternatively, a nucleic acid sequence can be synthesized having random nucleotides at one or more predetermined positions to generate random amino acid substituting.

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of ZEEP1 levels in cells and tissues may be effected by transforming the plant cells or tissues with at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

RNAi refers to the introduction of homologous double stranded RNA (dsRNA) to target a specific gene product, resulting in post transcriptional silencing of that gene. This phenomena was first reported in Caenorhabditis elegans by Guo and Kemphues (1995, Cell, 81(4):611-620) and subsequently Fire et al. (1998, Nature 391:806-811) discovered that it is the presence of dsRNA, formed from the annealing of sense and antisense strands present in the in vitro RNA preparations, that is responsible for producing the interfering activity.

The present invention contemplates the use of RNA interference (RNAi) to down regulate the expression of ZEEP1 in a plant to enhance the tolerance of the plant to abiotic stress. The dsRNA used to initiate RNAi, may be isolated from native source or produced by known means, e.g., transcribed from DNA. Plasmids and vectors for generating RNAi molecules against target sequence are now readily available as exemplified herein below.

The dsRNA can be transcribed from the vectors as two separate strands. Alternatively, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

According to certain embodiments, silencing the expression of ZEEP1 gene or a homolog thereof comprising transforming the cell with an RNAi molecule targeted to SEQ ID NO:20.

According to certain embodiments, transformation of the DNA constructs of the present invention into a plant cell is performed using Agrobacterium system.

The transgenic plant is then grown under conditions suitable for the expression of the recombinant DNA construct or constructs.

The regeneration, development and cultivation of plants from single plant protoplast transformants or from various transformed explants is well known in the art (Weissbach and Weissbach, In.: Methods for Plant Molecular Biology, (Eds.), 1988 Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The regenerated plants containing the foreign, exogenous gene that encodes a protein of interest or silence a gene of interest can then be further propagated as is well known in the art. The particular method of propagation will depend on the starting plant tissue and the particular plant species to be propagated. Preferably, the regenerated plants are self-pollinated to provide homozygous transgenic plants. Otherwise, pollen obtained from the regenerated plants is crossed to seed-grown plants of agronomically important lines, or pollen from plants of these important lines is used to pollinate regenerated plants. A transgenic plant of the present invention containing a desired polypeptide is cultivated using methods well known to one of skill in the art.

As exemplified hereinbelow, the transgenic plants of the present invention overexpressing the At5TPase7 and/or At5TPase9 genes show high tolerance to saline medium (salt concentration of 150-200 mM) and to drought conditions. Plants harboring a mutated ZEEP1 were also shown to be highly tolerant to salt, osmotic and water stress. Plants having increased tolerance to drought can easily adjust to growth under semi-dry and dry conditions, a trait which is highly desirable due to the growing process of desertification in agricultural areas all over the world.

As used herein, the term “salt concentration” refers particularly to “NaCl concentration”. However, it is to be understood that the teachings of present invention encompasses any equivalent salt that may be present in a plant growth medium, including, for example, KCl, and CaCl₂.

Also within the scope of this invention are seeds or plant parts obtained from the transgenic plants. Plant parts include differentiated and undifferentiated tissues, including but not limited to, roots, stems, shoots, leaves, pollen, seeds, tumor tissue, and various forms of cells and culture such as single cells, protoplasts, embryos, and callus tissue. The plant tissue may be in plant or in organ, tissue or cell culture.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

Material and Methods

Plant Material

Arabidopsis (Arabidopsis thaliana) ecotype Columbia (Col-0) plants were used as the wild type and as the background for the mutations and transgenic lines.

The mutants analyzed here (At5TPase7, At5TPase9) were from the Arabidopsis Biological Resource Center (Alonso J M et al., 2003. Science 301, 653-657), and At5PTase1 and At5PTase2 mutants were described by Gunesekera B et al. 2007. Plant Physiol 143, 1408-1417). Seeds were surface sterilized and germinated on one-half-strength Murashige and Skoog (MS) 0.8% (w/v) phytagar medium without sucrose. Prior to germination, seeds were stratified at 4° C. for 2 days and grown at 24° C. under constant or long-day-regime (16 h of light) fluorescent light (120 μE).

At5PTase7 (At2g32010) mutant lines Salk_{—}040226, Salk_{—}038842, and Salk_{—}038828; and At5PTase9 (salk_{—}090899) were verified by PCR as described at the Salk Institute Genomic Analysis Laboratory Web site (http://signal.salk.edu/tdnaprimers.2.html).

Cloning of At5TPases

Cloning of At5PTase7, was performed as described previously (Ercetin and Gillaspy, 2004, ibid). Briefly, full-length cDNAs were generated by RT-PCR using 1 mg of Arabidopsis seedling mRNA and the following primer combination: At5PTase7-Nterm (5′-GCCATGGTGGTGATTCTTGAGAAC-3′, SEQ ID NO:5) and At5PTase7-Cterm (5′-GAAAAATGTTAGCTCGGTGTATC-3′, SEQ ID NO:6). The cDNA products were gel purified and cloned into the pMT/V5-His-TOPO vector (Invitrogen).

For cloning At5PTase9, RNA was extracted, and the At5PTase9 coding region of 1,254 bp was converted to cDNA by the reverse transcriptase SuperScript III kit (Invitrogen). cDNA was amplified with the following primers: forward (5′-CACCATGTGGCCAAGACTTGTGGCC-3′, SEQ ID NO:21) and reverse (5′-CTATGTAGATATCCACGAGTAGTC-3′, SEQ ID NO:22). PCR was performed with PrimeSTAR HS DNA Polymerase (Takara), and cloned into the pENTR/D-TOPO vector using the pENTR directional TOPO Cloning Kit (Invitrogen K2400-20) and later into the binary Gateway 35S overexpression vector pB7WG2 using Gateway technology (Karimi M et al., 2002. Trends Plant Sci 7, 194-195).

Transformation of the vectors was done by Agrobacterium tumefaciens method, widely used for creating transgenic plants. Typically, plasmids were introduced into Agrobacterium tumefaciens strain GV3101 (Koncz C et al., 1989. Proc Natl Acad Sci USA 86: 8467-8471) and transformed by the floral dip procedure (Clough S J and Bent A F. 1998. Plant J 16, 735-743). Transgenic lines expressing high levels of At5PTase7 or At5PTase9 were propagated for three generations on kanamycin.

Cloning of At5PTase7-GFP

RNA was extracted, and the At5PTase7 coding region of 1,785 bp was converted to cDNA by the reverse transcriptase SuperScript III kit (Invitrogen). cDNA was amplified with the following primers: forward (5′-CACCATGAGAGACGATAAAACCAAGAAA-3′, SEQ ID NO:7) and reverse (5′-TTAGAAAAATGTTAGCTCGGTGTA-3′, SEQ ID NO:8). PCR was performed with PrimeSTAR HS DNA Polymerase (Takara), and cloned into the pENTR/D-TOPO vector using the pENTR directional TOPO Cloning Kit (Invitrogen K2400-20) and later into the binary Gateway 35S overexpression vector pK7WG2 or GFP vector pK7WGF2 using Gateway technology (Karimi M et al., 2002, ibid). The resulting plasmid was introduced into Agrobacterium tumefaciens strain GV3101 (Koncz C et al., 1989, ibid) and transformed by the floral dip procedure (Clough S J and Bent A F. 1998. ibid). Transgenic lines expressing high levels of At5PTase7 were propagated for three generations on kanamycin.

Transient Protoplast Transfection

Arabidopsis ecotype Columbia plants were grown in short-day conditions at 20° C. Leaves were collected 4 to 6 weeks after germination and used for the isolation and transformation of protoplasts as described (Yoo S-D et al., 2007. Nature Protoc 2, 1565-1572). Fluorescence was detected 24 h after transfection using the Zeiss LSM 510.

Real-Time RT-PCR

RNA was extracted and analyzed by RT-PCR as described (Leshem et al., 2007 ibid). The real-time PCR reactions contained 6 μl of cDNA, 1.5 μl of 3 mM primers, and 7.5 μl of DyNAmo FLASH SYBR Green qPCR kit (Finnzymes). The reaction was performed on a Rotor Gene 2000 thermocycler (Corbett Research). PCR conditions were as follows: 95° C. for 2 min; followed by 40 cycles of 95° C. for 15 s, 60° C. for 10 s, and 72° C. for 15 s; and then fluorescence acquisition at 79° C. PCR product specificity was verified by melting curve analysis and sequencing. For quantification, calibration curves were run simultaneously with experimental samples, and Ct (cycle number) calculations were performed by the Rotor-Gene 5.0 software (Corbett Research). The Actin2 gene was used for normalization. The following primers were used for the PCR:

For Actin2:
(SEQ ID NO: 9)
L, 5′-CTGCTTGGTGCAAGTGCTGTGATT-3′;
(SEQ ID NO: 10)
R, 5′-AGAAGTCTTGTTCCAGCCCTCGTT-3′,
For 5PTase2:
(SEQ ID NO: 11)
L, 5′-CGGTGATCGAACACTTCAACTCCA-3′;
(SEQ ID NO: 12)
R, 5′-TTCGATTCTGATCGTTACGCCGGA-3′,
For 5PTase3:
(SEQ ID NO: 13)
L, 5′-ATCGCTGTAACCGGACGATGATCT-3′;
(SEQ ID NO: 14)
R, 5′-TTCCGATGAGTACACCGCAAACGA-3′,
For 5PTase7:
(SEQ ID NO: 15)
L, 5′-TCAGAAAACCGTGACTCCCCT-3′;
(SEQ ID NO: 16)
R, 5′-GATGGAACGAAGGAAAGATATACT-3′;
For 5PTase11:
(SEQ ID NO: 17)
L, 5′-ACCCACTTCAAGCAAAGATCCGCT-3′;
(SEQ ID NO: 18)
R, 5′-GCCATAAGATCAGGGTTCCAGCTT-3′.

Plant Treatments

High Salinity

Seedlings were germinated on 150 mM NaCl or on one-half-strength MS agar plates and replanted onto plates supplemented with salt as described by Leshem et al. (2007, ibid). Alternatively, to bypass possible variation from the imbibition step, seeds were germinated on one-half-strength MS medium and transferred to plates containing 150 mM NaCl after seven days.

Water Stress

Examined plants (wild-type, At5ptase9, zeep1-1 or zeep1-2 mutants, plants overexpressing the At5TPase5 gene) were grown in pots filled with soil under standard growth conditions (16 hours daylight, 24° C., watered two times a week) for 5-6 weeks, and then irrigation was stopped (water stress or drought conditions). Appearance of wild-type, At5ptase mutants and plants overexpressing the At5TPase5 gene was examined after two weeks without water. Wild type, zeep1-1 and zeep1-2 mutant plants were left without irrigation until the signs of losing turgor appeared (after 5 days), and were then re-watered. Survival and appearance of the plants were measured after the second irrigation.

Reactive Oxygen Species (ROS) Analysis

ROS production was assayed with 10 μM 2′,7′-dichlorofluorescein (H₂DCFDA), which becomes trapped inside the cytoplasm following oxidation by H₂O₂. The fluorescence was observed by epifluorescent and by confocal microscopy as described below. Quantification of ROS was performed by ImagePro Plus analysis package (Media Cybernetics; MD, USA).

Analysis of Ca²⁺ Influx and Measurements

Intracellular Ca²⁺ was assayed by the Fluo-4-AM probe (Molecular Probes, Invitrogen, Carlsbad, Calif.). The dye (25 μM) was loaded in the presence of pluronic acid (Molecular Probes-Invitrogen, Carlsbad, Calif.) and washed. The levels of Ca²⁺ were quantified by ImagePro Plus analysis package (Media Cybernetics).

Endocytosis Assay

Internalization of the plasma membrane was assayed using the membrane tracking probe FM4-64 as described in Leshem et al. 2006 (Leshem Yet al., 2006. Proc. Natl. Acad. Sci. USA 103, 18008-18013). Briefly, seedlings were stained for 10 min, washed in ½ MS and observed by epifluorescence microscopy. The dye internalization was quantified by analyzing FM4-64 fluorescence using ImagePro Plus.

Confocal and Epifluorescence Microscopy

Confocal imaging was performed with an inverted Zeiss LSM 510 laser scanning microscope with a 203 air objective and a 403 or 633 oil-immersion objective. For imaging H₂DCFDA (ROS) alone or GFP together with 4′,6-diamidino-2-phenylindole (DAPI), the single-track and multiple-track facilities of the confocal microscope were used, respectively. For imaging GFP and ROS, the 488-nm excitation line was used. For the DAPI stain, the 405-nm excitation laser was used. Fluorescence was detected using a 505- to 550-nm band-pass filter for GFP and ROS stains. A 420- to 480-nm band-pass filter was used for the DAPI stain, and a 650-nm long-pass filter was used for the chlorophyll autofluorescence. Post-acquisition image processing was by LSM 5 Image Browser (Zeiss). In some experiments, an epifluorescence microscope (Olympus IX70) equipped with a narrow-band filter cube (excitation/emission, 485DF22/535DF35) from Omega was used. Images were taken with Coolpix 950 digital camera (Nikon) using identical exposure settings for each set of images, as described (Mazel et al., 2004, ibid).

PtdIns Treatments

PtdIns(3)P was purchased from Echelon Biosciences. The PtdIns were delivered into cells using carrier molecules according to the manufacturer's recommendations. The Shuttle carrier 2 was dissolved with PtdIns at a 1:1 ratio to form PtdIns:carrier complex, final 12.5 mM concentration, and incubated with the seedlings for 1 h before treatment with NaCl.

Expression and Immunoprecipitation of At5PTases from S2 Cells

Expression and immunoprecipitation of At5PTases from Drosophila S2 cells were performed as described (Ercetin and Gillaspy, 2004, ibid). Briefly, S2 cells were transfected with 2 μg of pMTAt5PTas constructs using an Effectene transfection kit (Qiagen). Cells were harvested after 2 d of induction with 500 μM CuSO₄. Immunoprecipitation of the At5PTases and analysis of the resulting complexes were performed as described (Ercetin and Gillaspy, 2004, ibid).

Activity Assays with Fluorescent and Radiolabeled Substrates

Activity assay conditions for fluorescent di-C₆-6(7Nitrobenz-2-oxa-1,3-diazol)-PtdIns-4,5-bisphosphate, di-C₆-6(7Nitrobenz-2-oxa-1,3-diazol)-PtdIns-3,4,5-trisphosphate, and ³H-labeled Ins(1,4,5)P₃ and Ins(1,3,4,5)P₄ were described before (Ercetin and Gillaspy, 2004, ibid). Recombinant At5PTases (15-80 ng) were incubated with 1.5 μg of fluorescent substrate in assay buffer containing 50 mM HEPES, pH 7.5, 5 mM MgCl2, and 50 mM KCl. All activity assays were performed for 1 h at room temperature. Reaction products were separated by thin-layer chromatography and analyzed as described before (Ercetin and Gillaspy, 2004, ibid).

### Example 1

### Expression of At5TPases Under Salt Stress Conditions

To analyze At5PTase gene expression in mature plants under different growth conditions, plants that grew in soil under normal conditions for 4 weeks were watered with 250 mM NaCl and RNA was extracted 6 h later. The expression of At5PTase2, At5PTase 3, At5PTase 7, and At5PTase 11 genes was analyzed by quantitative real-time reverse transcription (RT)-PCR. An induced transcription of the At5PTase7 gene in the mature plants grown in soil watered with saline solution was observed. Transcription of At5PTase2 and At5PTase 3 in mature plants was not induced (FIG. 1). Moreover, the transcription of the At5PTase11 gene in the roots of seedlings (1 week old) was up-regulated, while in mature plants the response was reversed, highlighting the dynamic functioning of At5PTases.

### Example 2

### Involvement of At5TPases in Arabidopsis Salt Tolerance

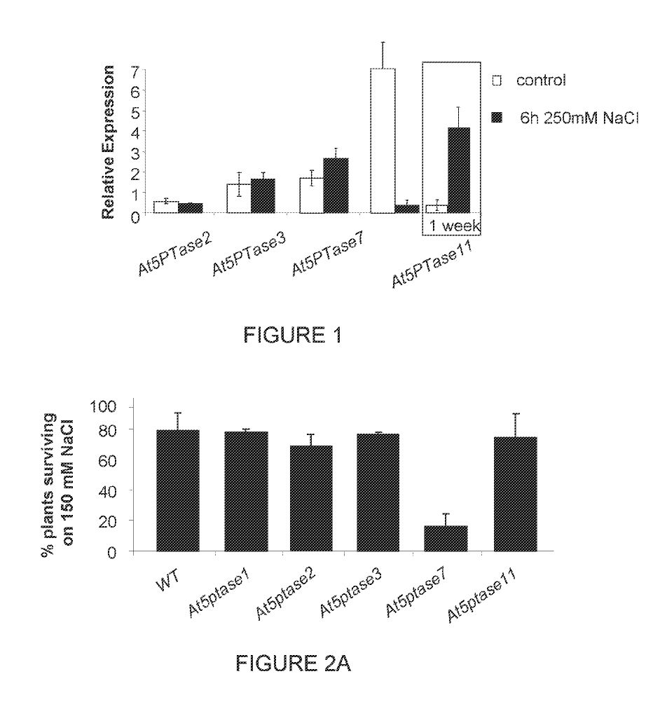

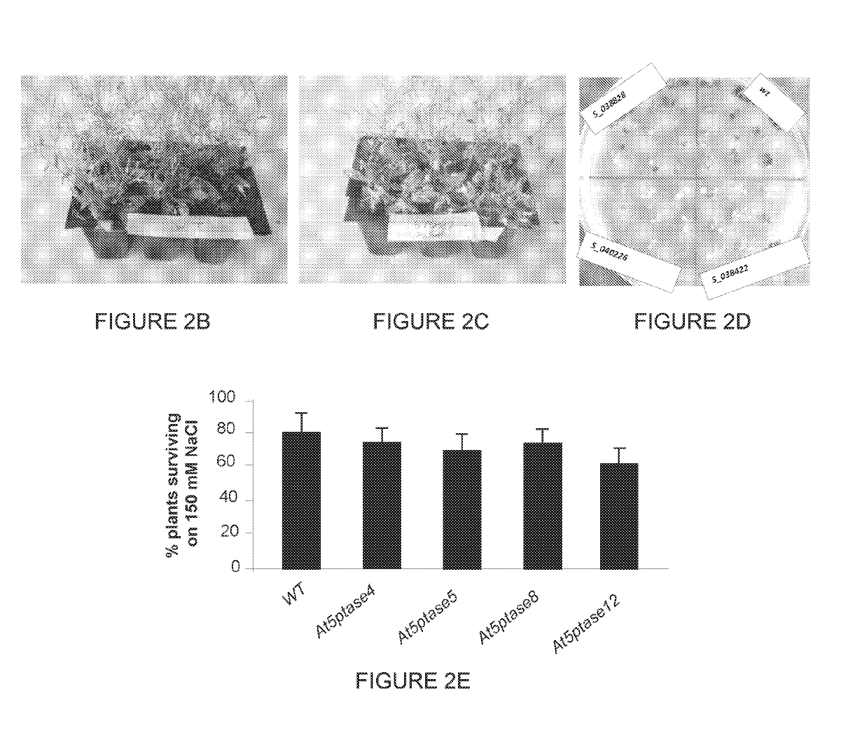

To analyze the physiological role of At5PTases in salt stress, the salt tolerance of several At5ptase mutants was assayed. Under normal (non-saline) conditions, the phenotypes of all mutants were similar to the wild-type plants. After 7 days, the sprouts were transferred to plates containing 150 mM NaCl. Such an abrupt increase in salinity, shortly after germination, is common in arid areas that necessitate irrigation of young seedlings with poor quality water right after plantation. The majority (four out of five) of homozygous mutant lines (At5ptase1, At5ptase 2, At5ptase 3, and At5ptase 11) looked similar to the wild type also during the salt stress. One mutant line, At5ptase7, exhibited a salt-overly-sensitive phenotype (FIG. 2A). The first symptom of salt stress in At5ptase7 mutants was bleaching of cotyledons, which occurred within 2 days, spreading to true leaves after another 1 to 2 days and resulting in death of the whole plant. In soil-grown 4-week-old plants, the stress symptoms developed slower, causing severe growth retardation, but not death of the whole plant (FIG. 2B—wilt type; FIG. 2C—At5ptase7 mutants). The mature wild type plants performed better than the At5ptase7 mutants. The salt sensitivity of the At5ptase7 mutants was reproduced in three different homozygous At5ptase7 mutant lines (Salk_{—}038828, Salk_{—}038842, and Salk_{—}040226, FIG. 2D). Five heterozygous At5ptase mutants were also analyzed, At5ptase2, At5ptase4, At5ptase 5, At5ptase8 At5ptase12. Most of these heterozygous mutants exhibited salt tolerance similar to the wild type, except At5ptase12 mutants that showed a slightly reduced tolerance (FIG. 2E). Importantly, all of the mutants, including At5ptase7 and At5ptase12, grew normally in control conditions, suggesting a specific function of these genes in response to salt stress.

In another experiment the salt stress responses of additional A. thaliana T-DNA mutants, namely At5ptase9 was analyzed, in comparison to the At5ptase1 and At5ptase2 mutants (FIG. 3A). The homozygous T-DNA insertion lines of At5ptases1 and At5ptases2 were obtained from ABRC (http://www.arabidopsis.org). The At5ptase9 T-DNA mutants were isolated from the Salk_{—}090899 line (FIG. 3B). Seeds were germinated on ½ strength MS medium (without sucrose) supplemented with 15 mM (control) or 150 mM NaCl. When grown in standard (non-saline) conditions the mutant lines were indistinguishable from wild-type plants. The At5ptase1 and At5ptase2 mutants did not show deviation from wild-type plants also on 150 mM NaCl. However, less than 10% of the At5ptase9 mutants (Salk_{—}090899) managed to germinate and survive for five days in the saline conditions (FIG. 3A). Interestingly, the At5ptase1 and At5ptase2 mutants were previously shown to have altered levels of Ins(1,4,5)P₃ in response to abscisic acid (ABA) treatment (Gunesekera et al., 2007 ibid).

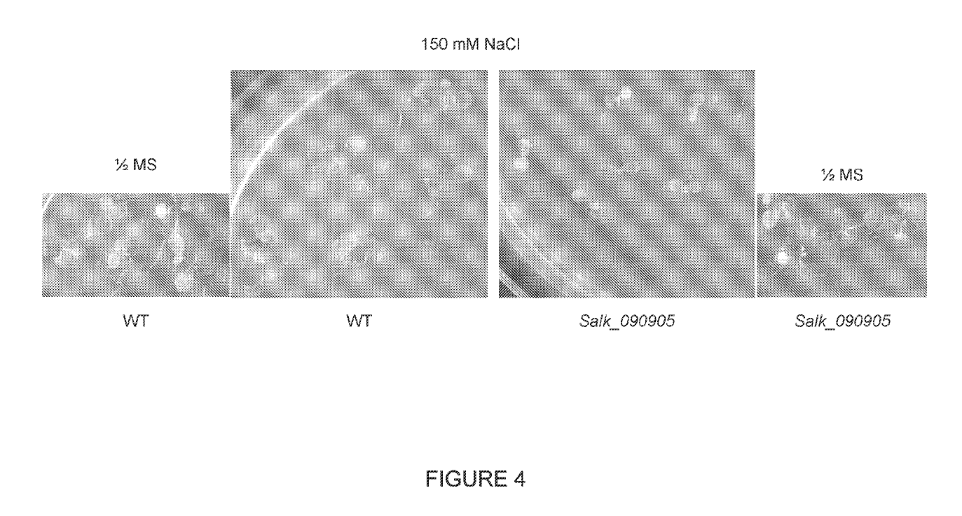

To bypass possible variation from the imbibition step, seeds were germinated on ½ MS medium and transferred to plates containing 150 mM NaCl after seven days. Transfer to high salinity resulted in acute bleaching of the At5ptase9 seedlings, but not in the wild type (FIG. 3C). Less than 5% of the homozygous At5ptase9 mutants survived for 5 days on 150 mM NaCl. Extreme salt sensitivity was also observed in another At5ptase9 mutant line, Salk_{—}090905 (FIG. 4). Interestingly, only 10% of the heterozygotic Salk_{—}090905 plants survived for 5 days on salt, indicating a haplo-insufficient nature of the allele.

Salt tolerance in mature plants was also examined. Plants were grown in soil and irrigated with tap water (control) and saline (supplemented with 150 mM NaCl) water. The mutants appeared normal when grown in pots under control conditions. Salt treatment caused bleaching in all plants after several days, but the effect was much more significant in the At5ptase9 mutants that collapsed completely after 7-10 days (data not shown).

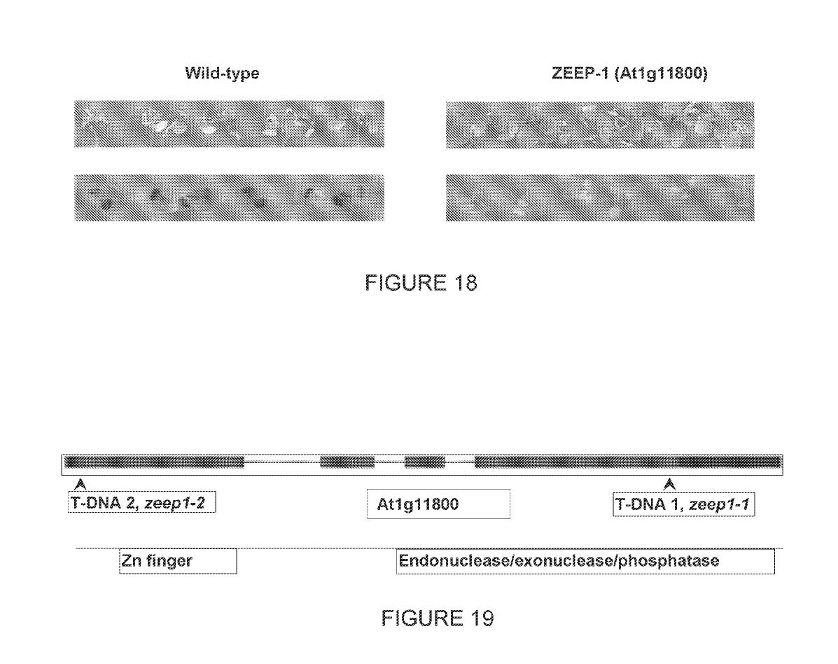

Unexpectedly, one of the mutants screened, At1g11800, showed improved salt tolerance compared to wild type. Seeds were germinated on ½ strength MS medium (without sucrose) supplemented with 150 mM NaCl. Sprouts of the mutant seeds showed increased survival and growth compared to sprouts of wild type seeds (FIG. 18). Improved slat tolerance of At1g11800 was also observed when this mutant was grown in a medium containing KCl, a salt which is known to cause ion stress similar to the stress caused by NaCl (Leshem et al. 2007, ibid).

### Example 3

### Overexpression of At5TPase

To test whether the expression level of the At5PTase7 or At5PTase9 genes influence salt and/or drought tolerance, the genes were cloned behind a constitutive 35S cauliflower mosaic virus promoter and introduced into wild-type Arabidopsis. Two At5PTase7 transgenic lines, identified to have the highest expression level by RT-PCR (OE-6 and OE-9 FIG. 5A), were selected for the experiment. The salt tolerance of the transgenic lines was tested by their germination and growth on medium containing 150 mM NaCl. More than 50% of the transgenic plants germinated and grew on salt, while none of the wild-type plants survived after 9 days (FIG. 5B). Transgenic lines overexpressing At5PTase9 also showed greatly improved germination and growth on saline (150 mM NaCl) medium (FIG. 3D)

Transgenic plants overexpressing the At5PTase9 gene were also examined for their tolerance to water stress (drought). Wild-type, At5ptase9 mutant and At5PTase9 overexpressing plants were grown in pots filled with soil under standard growth conditions (16 hours daylight, 24° C., watered two times a week) for 6 weeks, after which irrigation was stopped (FIG. 6A). Pictures were taken after 2 weeks without water. The watered control plants are shown in FIG. 6B. The At5PTase9 overexpressing plants were highly resistant to the water stress, showing equivalent growth to that of the wilt type watered control.

### Example 4

### Molecular Characterization of the At1q11800 Mutant

Homozygotic plants were isolated from the Salk_{—}043413 (Salk_{—}126721) T-DNA line (Alonso et al., 2003, ibid). Molecular analysis of the At1g11800 gene showed that in addition to the Endonuclease/Exonuclease/Phosphatase (EEP) domain, it contains a RanBP2 type Zinc Finger (ZnF) domain; the gene was thus designated ZEEP1 for Zinc Finger EEP. The ZEEP1 protein is a unique endonuclease/exonuclease/phosphatase in the A. thaliana genome that contains a ZnF domain in the N-terminus (FIG. 19). A very limited number of proteins that contain both, EEP and ZnF domains have been identified in other plants as well. For example, only one gene was identified in rice, Populus trichocarpa (California poplar) and castor oil plant (Ricinus communis), and two such gene were identified in grape vine (Vitis vinifera).

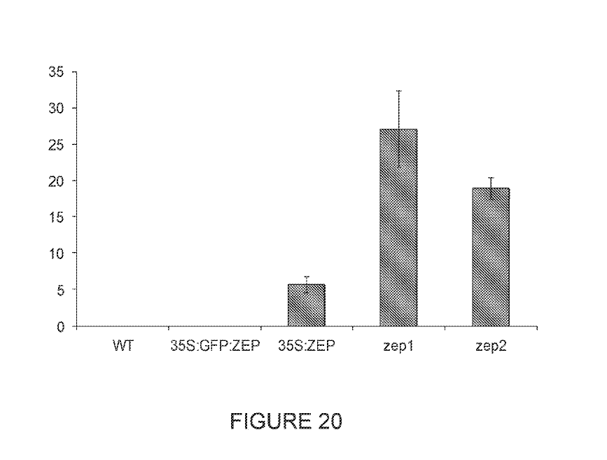

To substantiate the improved salt tolerance of mutants in the ZEEP1 gene the response of plants harboring another mutation in the same gene was examined. While the first mutation (zeep1-1) was within the EEP domain, the second mutation, zeep1-2, occurred at the ZnF domain (FIG. 19). First, germination on saline medium of both zeep1 mutants was assayed. Next, to bypass the imbibition step seeds were germinated in ½ strength MS medium and transferred to saline condition after 7 days. More than 50% of both mutants could grow on 150 mM NaCl for 7 days, while none of the wild type plants could grow in this salt concentration. Particularly tolerant were the zeep1-1 mutants (FIG. 20).

### Example 5

### Drought Tolerance of Zeep1 Mutants

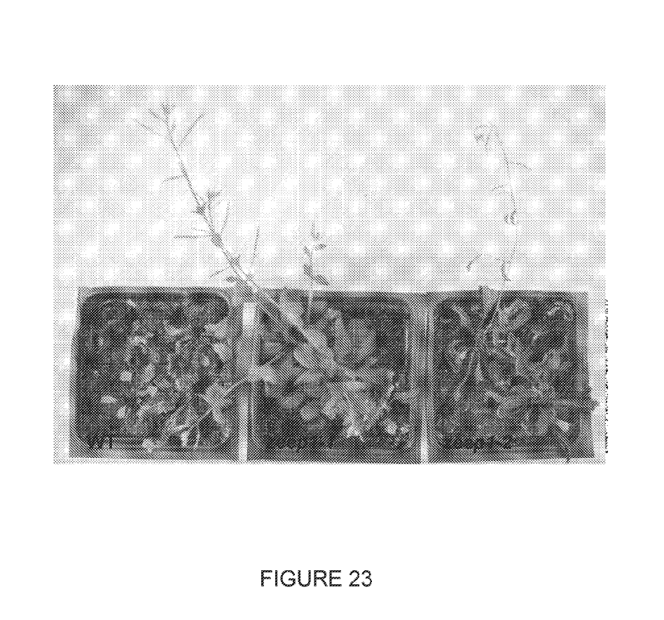

Seedlings of zeep1-1 and zeep1-2 mutants were transferred to soil after seven days in petri plates and grown in constant watering (twice a week) regime under long-day (16 h light) conditions. After 5 weeks the irrigation was discontinued, and the plants were allowed to wilt. When the plants began to show signs of losing turgor (which occurred after five days) they were re-watered. The zeep1-1 mutants and partially the zeep1-2 mutants regained the turgor, while the wild-type plants continued to wilt and soon died (FIG. 23).

### Example 6

### Tissue and Cellular Localization of At5PTase Proteins in Arabidopsis

Localization of a protein is an important attribute of its function, especially for proteins involved in intracellular vesicle trafficking. Hence, the At5TPase7 and At5TPase9 genes were fused to marker genes enabling detecting the gene expression within the plant cells.

At5TPase7

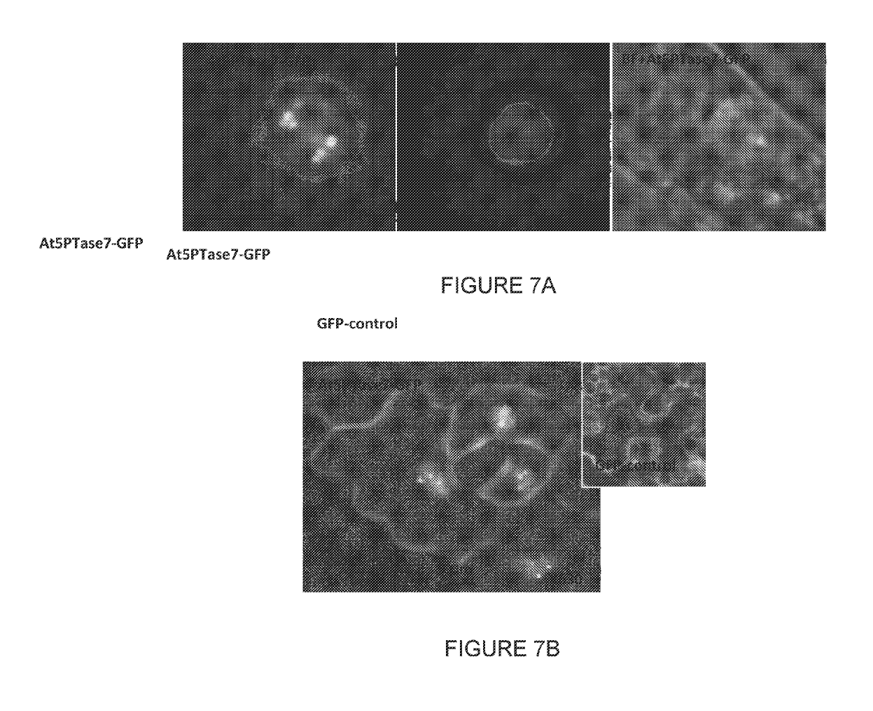

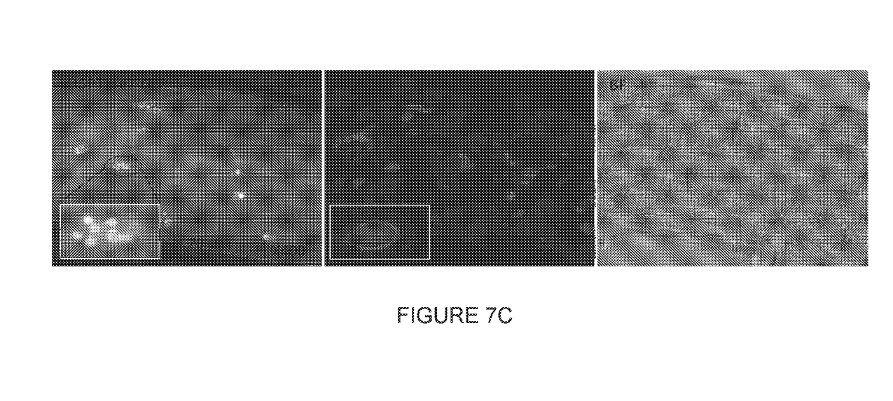

At5TPase7 was fused to GFP and its localization was examined in protoplasts and tissues of transgenic plants by epifluorescence and confocal microscopy. The At5PTase7-GFP protein was detected in the proximity of the plasma membrane and in the nuclei of transgenic plants in both roots and leaves (FIG. 7) as well as in protoplasts (data not shown). Strikingly, At5PTase7-GFP appeared as distinct nuclear speckles, which are thought to constitute sub-nuclear structures enriched in pre-mRNA splicing factors of the nucleoplasm (Lamond A I and Spector D L. 2003. Nat Rev Mol Cell Biol 4, 605-612). The At5PTase7-GFP-containing speckles were observed in transiently transfected protoplasts as well as in the nucleoplasm of the transgenic plants (FIGS. 7B and C). The nuclear localization of At5PTase7-GFP is in agreement with the SubLoc program for protein sub-cellular location based on the Simple Object Access Protocol (Heazlewood J L et al., 2005. Plant Physiol 139, 598-609), which is associated with The Arabidopsis Information Resource database.

At5TPase9

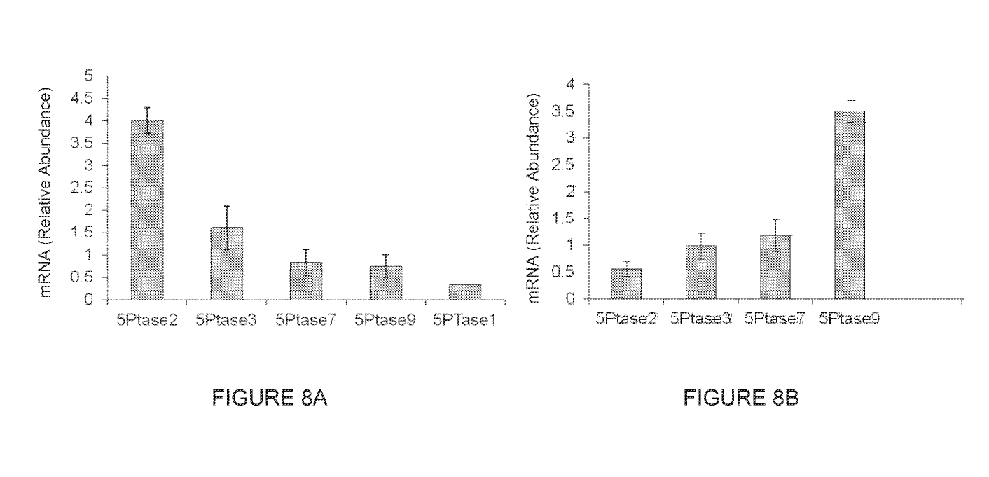

To analyze the tissue-specific expression of At5PTase9, an At5PTase9-GUS fusion construct was prepared, attached to the At5PTase9 natural promoter, including ˜1000 base pairs upstream of the transcription start site. The construct was introduced into wild-type Arabidopsis thaliana. The seedlings were stained with X-GlucA after seven days, washed in 50% ethanol (to clear the chlorophyll), and observed by light microscopy. Strong GUS staining was observed only in the roots (FIG. 8B). The root-specific expression was confirmed by RT-PCR in mature plants that were grown in pots for 3.5 weeks. Interestingly, such potent expression pattern in the roots was observed for the At5PTase9 gene, and to a lesser extent for the other stress-related gene, At5PTase7 that was expressed both in roots and shoots. The other At5PTase mutants showed an opposite, shoot-specific expression pattern, particularly for the At5PTase2 gene (FIG. 8).

ZEEP1

The ZEEP1 protein was fused (in-frame) to GFP, and the sub-cellular localization of ZEEP1 in A. thaliana protoplasts was analyzed by confocal microscopy. A strong fluorescent signal was detected in the nucleus, which overlapped with nuclear staining by DAPI. Addition of 200 mM NaCl resulted in dispersal of the GFP fluorescence within the cytoplasm, with no remaining signal in the nucleus, suggesting that ZEEP1 is translocated from the nucleus during salt stress.

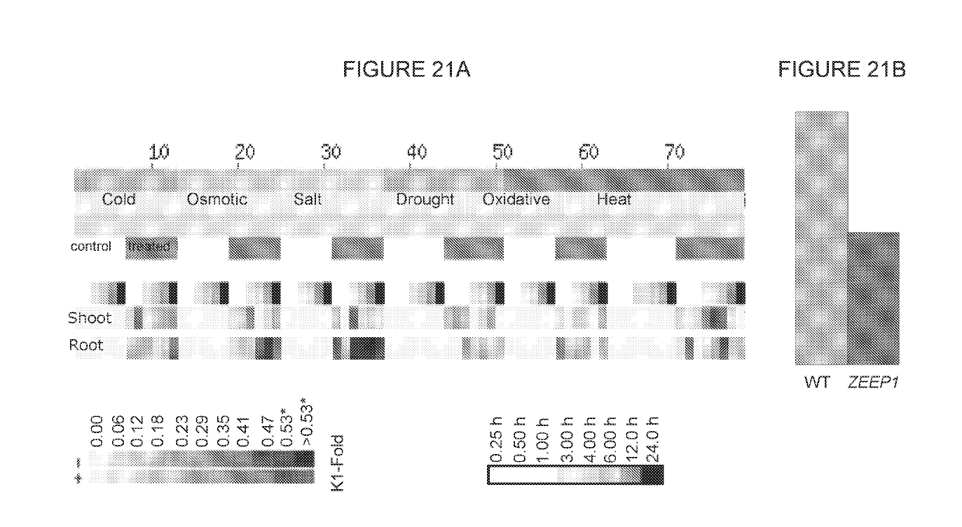

To attain a broader view on the involvement of ZEEP1 in cellular activities, its gene expression during common environmental stresses was analyzed, based on data from Bio-Array Resource for Arabidopsis Functional Genomics site (http://bbc.botany.utoronto.ca). An interesting expression pattern was observed in the roots. The gene was induced by cold and by drought, but it was strongly suppressed by salt and after a brief (1 h) induction by osmotic stress (FIG. 21A). Drought and oxidative stresses induced the At1g11800 gene expression very briefly. Analysis of ZEEP1 expression in mature plants showed that it maintained the down regulation (FIG. 21B).

### Example 7

### Reactive Oxygen Species (ROS) Production in At5ptase Mutants During Salt Stress.

A major problem associated with salt stress is the development of a secondary oxidative stress (Smirnoff N. 1998, ibid). Previous studies showed that ROS production during salt stress is caused by NADPH oxidase. Recently, it was shown that activation of NADPH oxidase in neutrophils, as well as in A. thaliana, is mediated by Phosphatidylinositol 3-kinase (Babior B M. 2004. Curr. Opin. Immunol. 16, 42-47; Leshem et al., 2007, ibid). Moreover, activation of the NADPH oxidase in neutrophils was showed to be depended on activity of a type II 5PTase (Babior, 2004, ibid).

ROS accumulation was measured in root tip and transition zone 15 min after the beginning of salt stress in At5ptase3, At5ptase7 and At5ptase11 mutants and in root tip and elongation zones 10 min after salt stress in At5ptase1, At5ptase2 and At5ptase9 mutants as well as in transgenic Arabidopsis plant over expressing At5ptase9.

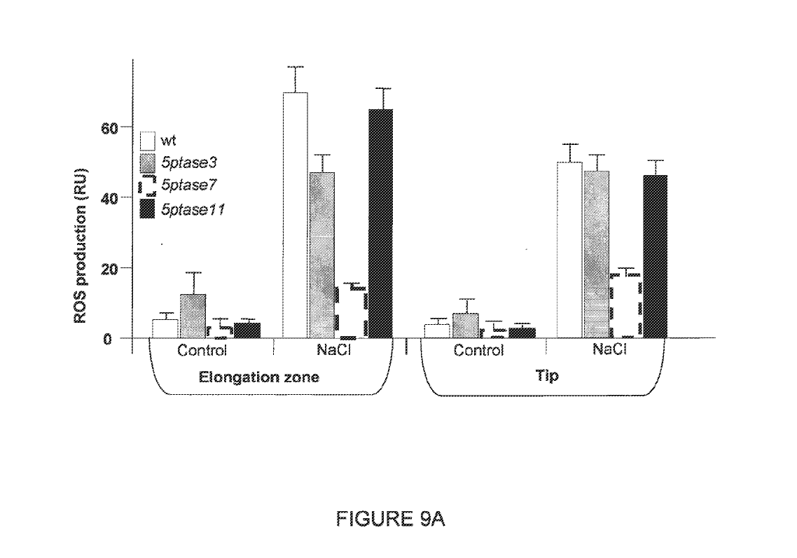

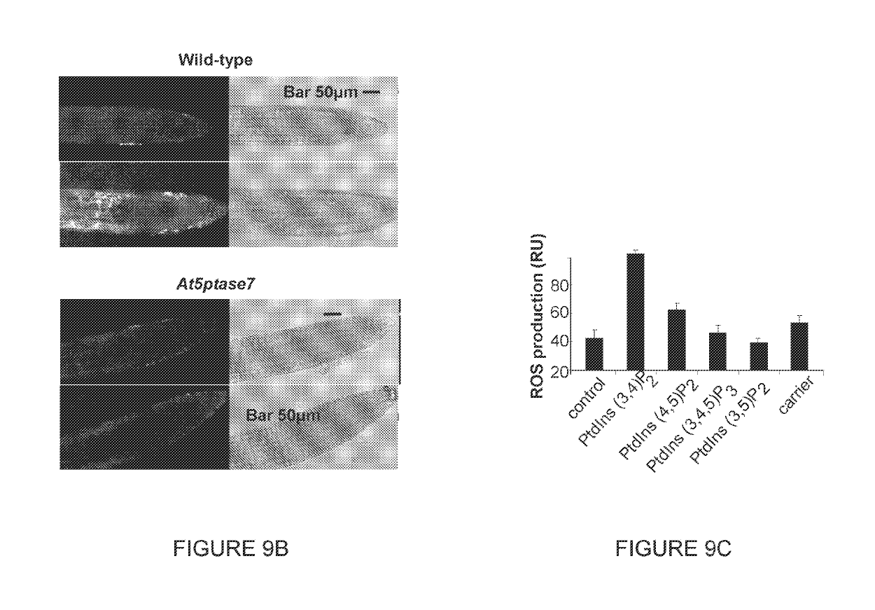

Normal ROS production was seen in At5ptase11 mutants, and a slightly lower ROS level was observed in the root elongation zone but not in the root tip of At5ptase3 under salt stress. However, very little ROS was detected in both regions of the At5ptase7 mutants (FIG. 9A-B). In additional experiment, ROS started to accumulate within minutes of NaCl addition in wild type as well as in At5ptase1 and At5ptase2 mutants but not in At5ptase9 mutant, both in the root tip and the elongation zone.

Therefore, it was examined whether ROS production in At5ptase7 mutants depended on phosphorylation of the D5′ position. It has been previously shown that exogenous PtdInsPs are taken up and delivered to correct sub-cellular locations in both animal and plant cells, including Arabidopsis seedlings. Accordingly, mutant seedlings were supplemented with differentially phosphorylated exogenous PtdInsPs, and the production of ROS was measured by confocal microscopy. The At5ptase7 mutants were preloaded with PtdIns(3,4)P₂, PtdIns(3,5)P₂, PtdIns(4,5)P₂, or PtdIns(3,4,5)P₃ and stimulated with 200 mM NaCl. The production of ROS was restored in seedlings supplemented with PtdIns(3,4)P₂ but not with PtdIns(4,5)P₂, PtdIns(3,5)P₂, or PtdIns(3,4,5)P₃ (FIG. 9C).

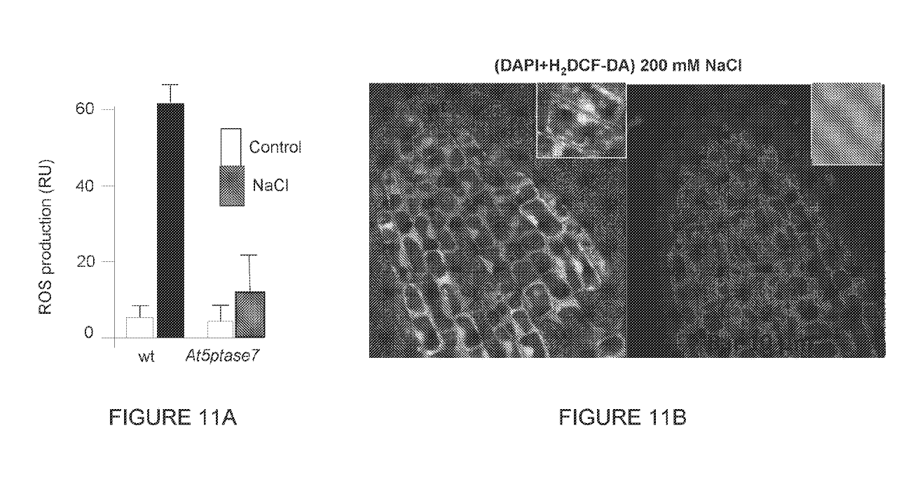

These results are in line with similar results in animal cells and in pi3K Arabidopsis mutants. Without wishing to be bound by any specific theory or mechanism of action, the results presented herein show that the activation of NADPH oxidase in Arabidopsis also requires conserved PtdIns phosphorylation sites, namely phosphorylation of the D3′ position and dephosphorylation of D5′. Recently, it was shown that ROS localization plays an important role in downstream signaling in plants as well as in animals. Therefore, the sub-cellular distribution of ROS was analyzed by confocal microscopy. ROS were detected throughout the cytoplasm as speckles that were primarily concentrated close to the plasma membrane (FIG. 11B Insert). Interestingly, a very definite ROS accumulation was detected in the nuclei in the roots of wild type plants (FIG. 11A). Nuclear ROS production in wild-type plants was very rapid, becoming visible in less than 10 min after exposure to salt. However, very little ROS were measured in the nuclei of At5ptase7 mutants (FIG. 11B).

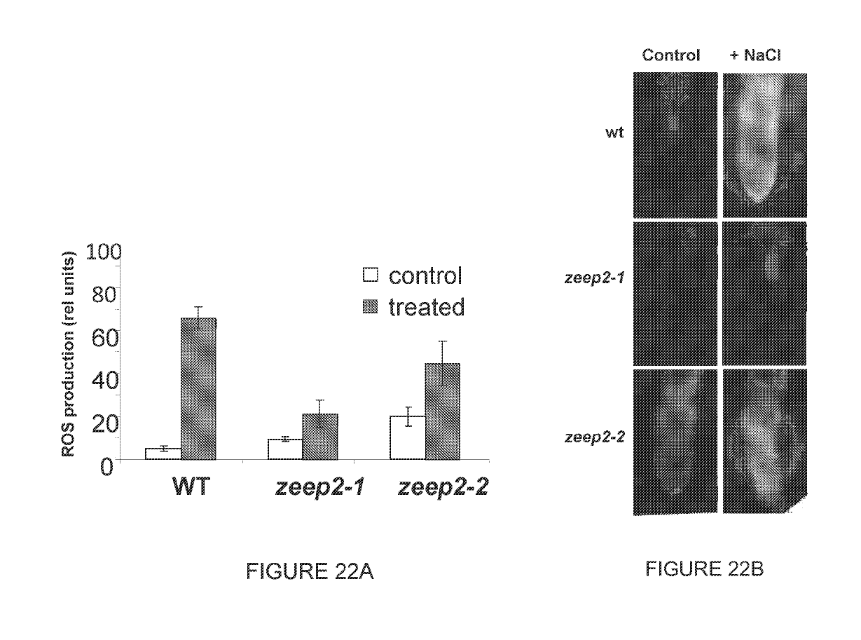

ROS production in plants harboring the zeep1 mutant, which show increased tolerance to abiotic stress, was also examined. zeep1 mutant seedlings were stained with H₂DCFDA that is sensitive to intracellular H₂O₂. ROS accumulation was visualized by epifluorescent and confocal microscopy and quantified by Image-Pro. Under control conditions all seedlings exhibited low production of ROS. Salt treatment caused a sharp increase in ROS accumulation in wild-type plants, but very little ROS production was seen in the zeep1-1 mutants, while the zeep1-2 mutants showed intermediate levels (FIG. 22A-B).

### Example 8

### Ca²⁺ Influx and ROS Accumulation

Numerous studies have shown that ROS production in plants is closely linked to calcium influx, so that both processes positively influence each other. To analyze whether the salt-induced ROS affect the influx of Ca²⁺ into the cytosol (Ca²⁺_{cyt} influx), ROS production in wild-type plants was inhibited during salt stress by the addition of the NADPH oxidase inhibitor diphenylene iodonium (DPI). Addition of the inhibitor abolished the stress-induced Ca²⁺_{cyt} influx (FIG. 12, insert), indicating that ROS production is essential for Ca²⁺_{cyt} influx. This result is in agreement with the reciprocal stimulation of Ca²⁺_{cyt} influx by H₂O₂ (Levine A et al., 1996. Curr. Biol. 6, 427-437).

The cytosolic Ca²⁺ levels were also examined in the At5ptase1, At5ptase2 and At5ptase9 mutants during salt stress. A rapid elevation of Ca²⁺_{cyt} was observed in the At5ptase1 and At5ptase2 mutants, which was comparable to wild-type plants following the salt treatment. However, no increase in the Ca²⁺ in the At5ptase9 mutants could be detected (FIG. 12).

### Example 9

### Reduced Fluid-Phase Endocytosis in At5ptase9 Mutants

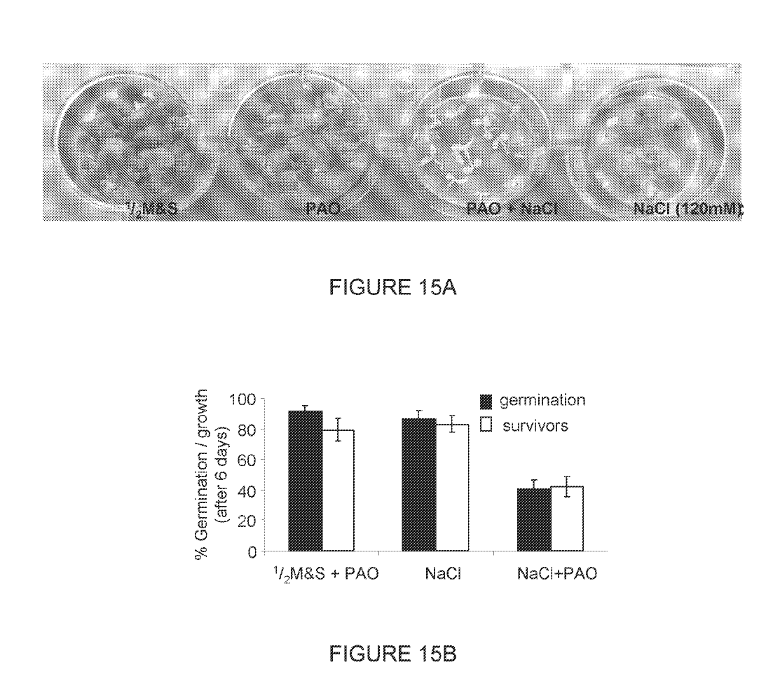

One mechanism that has been shown to be involved in the regulation of ROS production and calcium influx is endocytosis. Moreover, endocytosis was recently implicated in the activation of NADPH Oxidase and ROS production (Miller F J et al., 2010. Antioxidants & Redox Signaling 12, 583-593). To test the relation between the production of ROS (FIG. 10) and Ca²⁺ influx (FIG. 11) with endocytosis, the rate of fluid-phase endocytosis (also called bulk-flow endocytosis) was assayed in At5ptase1, At5ptase2 and At5ptase9 mutants by measuring the internalization of a membrane dye, FM4-64 (Leshem Y et al., 2006. ibid). Uptake of the FM4-64 dye by At5ptase1 and At5ptase2 mutants was similar or even higher compared to wild-type. However, a significantly lower internalization was detected in the At5ptase9 mutants (FIG. 13). The correlation observed between the FM4-64 uptake, influx of Ca²⁺_{cyt} and ROS accumulation suggested a possible regulation by endocytosis. In order to investigate this option, endocytosis in wild-type plants was inhibited by two chemically different types of inhibitors, Tyrphostin A23 (TyrA23) or Phenylarsine oxide (PAO) (Aniento F and Robinson D G. 2005. Protoplasma 226, 3-11; Robinson D G et al., 2008. Plant Physiol. 147, 1482-1492). Both inhibitors sharply decreased the dye accumulation in the cytoplasm, particularly TyrA23 (FIG. 13, right bars). Analysis of the inhibitors' effect on salt-induced influx of Ca²⁺_{cyt} and ROS generation showed that they completely blocked ROS production (FIG. 14A), as well as calcium influx (FIG. 14B). Thus, inhibition of endocytosis in wild-type plants mimicked the salt stress responses, described here for the At5ptase9 mutants. Moreover, inhibition of endocytosis decreased the salt tolerance in wild-type plants (FIG. 15).

### Example 10

### Substrate Preferences of At5PTases

In general, the 5PTase enzymes are capable of hydrolyzing both membrane bound PtdInsPs and/or soluble InsPs. To understand the molecular function of individual At5PTases it is important to determine which InsP and PtdInsP substrates they hydrolyze. The substrate preference of At5PTase7 was compared with the previously characterized enzymatic activity of At5PTase1, At5PTase2 and At5PTase3 (Berdy S et al., 2001. Plant Physiol 126, 801-810). Proteins were expressed as recombinant proteins in a Drosophila S2 cell system that has been previously shown to yield active 5PTase enzymes (Ercetin and Gillaspy, 2004, ibid).

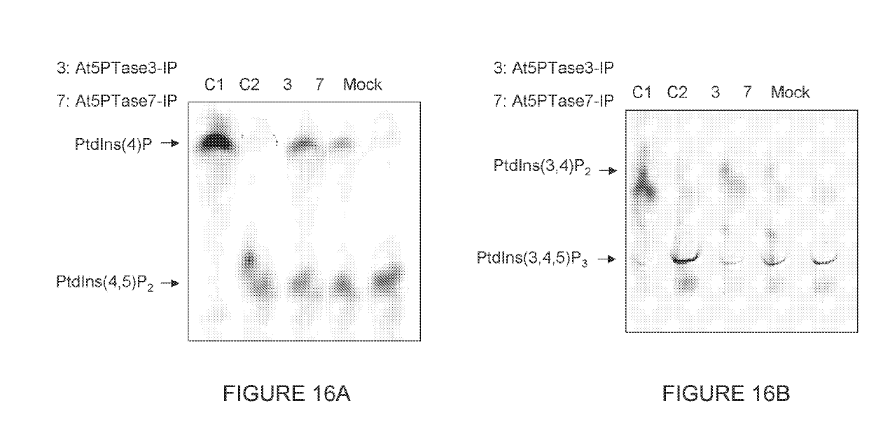

Recombinant enzymes were immunoprecipitated and incubated in activity assay with fluorescent substrates including PtdIns(4,5)P₂ and PtdIns(3,4,5)P₃. At5PTase3, known to hydrolyze PtdIns(4,5)P₂ and PtdIns(3,4,5)P₃, was used as a positive control (FIG. 16). Reaction products were separated by thin-layer chromatography. At5PTase7 hydrolyzed both PtdIns(4,5)P₂ and PtdIns(3,4,5)P₃, albeit not with the same efficiency (FIG. 16). Note that the low-abundance band in PtdIns(3,4)P₂ and PtdIns(3,4,5)P₃ standards is most likely due to PtdInsP₃ contamination of the commercial standards. The substrate preference of At5TPase9 was measured by the same procedure and was also found to hydrolyzed both PtdIns(4,5)P₂ and PtdIns(3,4,5)P₃. Since water-soluble substrates such as Ins(1,4,5)P₃ can also be hydrolyzed by certain 5PTases, a second activity assay was used to measure hydrolysis of this substrate. Immunoprecipitated At5PTase1, At5PTase2, At5PTase7 and At5PTase9 were each incubated with [³M-Ins(1,4,5)P₃ and HPLC was used to separate the Ins(1,4,5)P₃ substrate and Ins(1,4)P₂ product in each reaction (data not shown).

It was previously shown that At5PTase1 and At5PTase2 can remove a phosphate from Ins(1,4,5)P₃ in these assays (Berdy et al., 2001, ibid) and this capability was verified under the current reaction conditions where a conversion of 25% of the [³M-Ins(1,4,5)P₃ substrate was measured. However no hydrolysis of [³M-Ins(1,4,5)P₃ was detected when At5PTase7 or At5PTase9 was incubated with [³M-Ins(1,4,5)P₃. It is thus concluded that At5PTase9 and At5PTase7 have a substrate preference similar to that of the previously characterized At5PTase11 (Ercetin and Gillaspy, 2004, ibid), with activity only on lipid-containing substrates such as PtdIns(4,5)P₂ and PtdIns(3,4,5)P₃.

### Example 11

### Regulation of Salt-Induced Gene Expression by At5PTase9

The phosphatidylinositides have been described in the plant and animal literature mainly in connection with the structure and function of plasmalemma or endomembranes, but recently PtdIns were detected in the nucleus, in regulation of gene expression. The expression of several salt-induced marker genes (RD29A, RD22, CBF2 and ICE1) were examined in wild-type and At5ptase9 mutants.

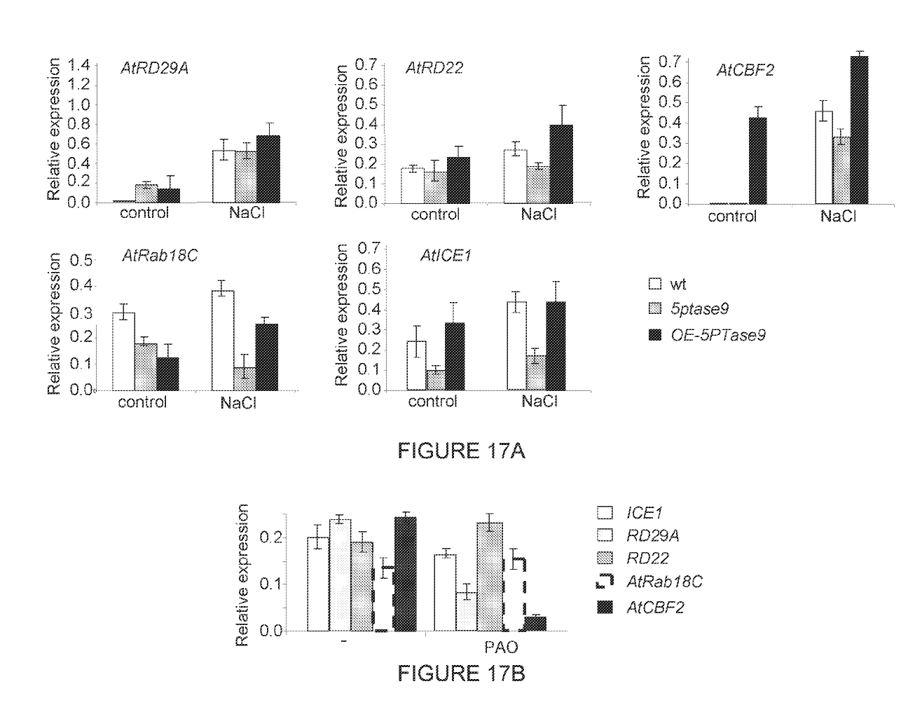

Strong induction in both, wild-type and mutant plants was seen in expression of RD29A gene. However, significantly smaller induction was seen in At5ptase9 mutants in RD22, ICE1, AtRab18C and CBF2 genes (FIG. 17A). Interestingly, overexpression of the At5PTase9 gene caused induction of the CBF2 transcription factor, even without NaCl treatment, indicating a direct effect on the CBF2 promoter activity (FIG. 16A, black bar). To test whether endocytosis acts in the induction of salt-responsive genes pathway, plants were pretreated with PAO for 1 hour and then stimulated by salt. PAO treatment reduced expression of RD29A, ICE1 and especially CBF2 genes, but not RD22 and AtRab18C genes (FIG. 17B).

### Example 12

### Regulation of Salt-Induced Gene Expression by ZEEP1

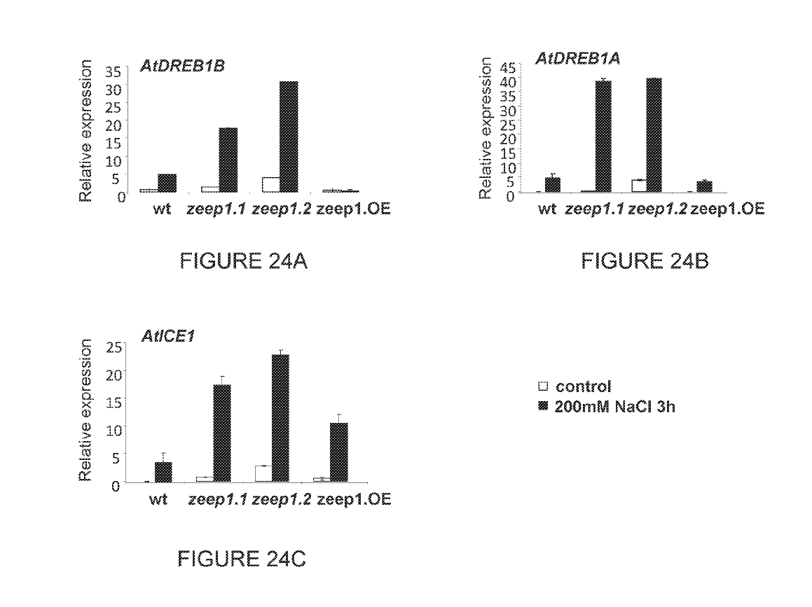

Without wishing to be bound by any specific theory or mechanism of action, translocation of ZEEP1 from the nucleus during salt stress suggests that it functions as a negative regulator of salt-responsive genes. To examine whether the nuclear translocation of ZEEP1 coincided with diminished expression of other stress-responsive genes, the expression of several common abiotic stress genes, including DREB1A, DREB1B and ICE1 was examined in wild-type and in zeep1 mutants. Given the more rapid response in the roots, the roots were separated from the shoots. Salt stress caused increased transcription of all selected marker genes in wild-type and mutant plants. However, in the zeep1 mutants the expression of DREB1B, DREB1A and ICE became slightly induced even without addition of salt (FIG. 23A-C). These results suggest that ZEEP1 functions as a negative transcriptional regulator, similar to the C2H2 type of Zinc finger proteins (Ciftci-Yilmaz and Mittler, 2008, ibid). To corroborate the negative role of the ZEEP1 protein on gene expression transgenic plants that overexpressed ZEEP1 were also produced and the transcription of stress marker genes during normal and salt stress conditions was analyzed. Interestingly, overexpression of the ZEEP1 suppressed the expression of DREB1B and DREB1A genes, further supporting the negative effect of ZEEP1 on gene induction (FIG. 24A-B).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A genetically modified plant comprising at least one cell having altered expression of at least one of Endonuclease/Exonuclease/Phosphatase (EEP) protein corresponding to any one of Arabidopsis thaliana 5TPase7 (At5TPase7), Arabidopsis thaliana 5TPase9 (At5TPase9) and Arabidopsis thaliana ZEEP1, wherein the plant has increased tolerance to environmental abiotic stress compared to a corresponding unmodified plant.

2. The genetically modified plant of claim 1, wherein said plant has an enhanced expression of at least one of At5TPase7, At5TPase9 or homologs thereof.

3. The genetically modified plant of claim 2, wherein said plant is a transgenic plant comprising at least one cell transformed with an isolated polynucleotide encoding at least one of At5TPase7 or a homolog thereof, At5TPase9 or a homolog thereof or a combination thereof, wherein the transgenic plant has increased tolerance to environmental abiotic stress compared to a corresponding non-transgenic plant.

4. The transgenic plant of claim 3, wherein the At5TPase7 has the amino acid sequence set forth in SEQ ID NO:1 or a homolog thereof and the At5TPase9 has the amino acid sequence set forth in SEQ ID NO:3 or a homolog thereof.

5. The transgenic plant of claim 3, wherein the polynucleotide encoding At5TPase7 has the nucleic acid sequence set forth in SEQ ID NO:2 and the polynucleotide encoding At5TPase9 has the nucleic acid sequence set forth in SEQ ID NO:4.

6. The transgenic plant of claim 3, wherein expression of the polynucleotide encoding At5TPase7 or the polynucleotide encoding At5TPase9 is controlled by a promoter selected from the group consisting of said plant 5TPase natural promoter, a constitutive promoter and a tissue specific promoter.

7. The genetically modified plant of claim 1, the plant having inhibited expression of ZEEP1 or a homolog thereof compared to the expression of ZEEP1 or its homolog in a corresponding unmodified plant.

8. The genetically modified plant of claim 7, wherein the ZEEP1 protein comprises the amino acid sequence set forth in SEQ ID NO:19 encoded by a polynucleotide having the nucleic acid sequence set forth in SEQ ID NO:20.

9. The genetically modified plant of claim 7, wherein said plant comprises at least one cell comprising a mutated ZEEP1 gene or a homolog thereof.

10. The genetically modified plant of claim 9, wherein the mutation is within the endonuclease/exonuclease/phosphatase (EEP) domain of the ZEEP1 gene.

11. The genetically modified plant of claim 7, wherein said plant is a transgenic plant comprising at least one cell comprising a ZEEP1 silencing molecule selected from the group consisting of RNA interference molecule and antisense molecule, wherein the silencing molecule is targeted to the ZEEP1 gene having the nucleic acid sequence set forth in SEQ ID NO:20 or a homolog thereof.

12. The genetically modified plant of claim 1, wherein the environmental abiotic stress is selected from the group consisting of soil salinity, drought (water stress), cold stress, heat stress, excess of light, presence of heavy metals or a combination thereof.

13. The genetically modified plant of claim 1, wherein the abiotic stress is selected from the group consisting of water content of less than 70% and soil salinity of above 4 dS/m.

14. The genetically modified plant of claim 1, wherein said plant is a crop plant selected from the group consisting of plants producing fruit; flower and ornamental plants; grain producing plants crops including wheat, oats, barley, rye, rice and maize; legumes including peanuts, peas soybean and lentil; forage crops used for hay or pasture; root crops; fiber crops; trees for wood industry; tuber crops sugar crops; and oil crops including canola, sunflower and sesame.

15. A seed of the plant of claim 1, wherein a plant grown from said seed comprises at least one cell having altered expression of at least one of Endonuclease/Exonuclease/Phosphatase (EEP) protein corresponding to any one of Arabidopsis thaliana 5TPase7 (At5TPase7), Arabidopsis thaliana 5TPase9 (At5TPase9) and Arabidopsis thaliana ZEEP1, and have enhanced tolerance to environmental abiotic stress compared to a plant grown from seeds of corresponding unmodified plant.

16. A tissue culture comprising at least one genetically modified cell of the plant of claim 1 or a protoplast derived therefrom, wherein said tissue culture regenerates a plant having at least one cell having altered expression of at least one of Endonuclease/Exonuclease/Phosphatase (EEP) protein corresponding to Arabidopsis thaliana 5TPase7 (At5TPase7), Arabidopsis thaliana 5TPase9 (At5TPase9) and Arabidopsis thaliana ZEEP1, and having enhanced tolerance to environmental abiotic stress compared to a plant regenerated from tissue culture of a corresponding unmodified plant.

17. A method for increasing the tolerance of a plant to environmental abiotic stress, the method comprising the steps of:
(a) transforming a plant cell with at least one isolated polynucleotide encoding At5TPase7, At5TPase9, homologs thereof or a combination thereof; and
(b) regenerating the transformed cell into a transgenic plant comprising at least one cell expressing At5TPase7, At5TPase9, homolog thereof or a combination thereof having an increased tolerance to environmental abiotic stress compared to a corresponding non-transgenic plant.

18. The method of claim 17, wherein the At5TPase7 has the amino acid sequence set forth in SEQ ID NO:1 or a homolog thereof and the At5TPase9 has the amino acid sequence set forth in SEQ ID NO:3 or a homolog thereof.

19. A method for increasing the tolerance of a plant to environmental abiotic stress, comprising (a) silencing the expression of ZEEP1 gene or a homolog thereof within at least one plant cell; and (b) regenerating the at least one plant cell comprising the silenced gene into a genetically modified plant having an increased tolerance to environmental abiotic stress compared to a corresponding unmodified plant.

20. The method of claim 19, wherein the ZEEP1 gene has a nucleic acid sequence as set forth in SEQ ID NO:20.
